(19) United States
(12) Patent Application Publication
(10) Pub. No.: US 2018/0280374 A1
(43) Pub. Date: Oct. 4, 2018

(54) PHARMACEUTICAL COMPOSITIONS

(71) Applicant: ADEPTIO PHARMACEUTICALS LIMITED, London (GB)

(72) Inventors: Andrew John DUFFIELD, Berkhamsted (GB); Anant PANDYA, Croydon (GB)

(73) Assignee: ADEPTIO PHARMACEUTICALS LIMITED, London (GB)

(21) Appl. No.: 15/939,822

(22) Filed: Mar. 29, 2018

Related U.S. Application Data
(60) Provisional application No. 62/515,937, filed on Jun. 6, 2017.
(60) Provisional application No. 62/515,928, filed on Jun. 6, 2017.

(30) Foreign Application Priority Data
Apr. 1, 2017 (GB) 1705301.8
Apr. 28, 2017 (GB) 1706816.4

(51) Int. Cl. A61K 31/473; A61P 25/14

(52) U.S. Cl. CPC A61K 31/473; A61K 31/13; A61K 2300/00; A61P 25/14

(57) ABSTRACT

This invention relates to the use of low doses of (+)-α-dihydrotetrabenazine for the treatment of movement disorders, such as Tourette's syndrome.

### CROSS REFERENCE TO RELATED APPLICATIONS

This application claims priority to U.S. Provisional Application No. 62/515,937, filed on Jun. 6, 2017, to U.S. Provisional Application No. 62/515,928, filed on Jun. 6, 2017, to Great Britain Application No. 1706816.4, filed on Apr. 28, 2017, and to Great Britain Application No. 1705301.8, filed on Apr. 1, 2017. The entire contents of each of the prior applications are hereby incorporated herein by reference.

This invention relates to the use of low doses of (+)-α-dihydrotetrabenazine for the treatment of movement disorders, such as Tourette's syndrome.

### BACKGROUND OF THE INVENTION

Movement disorders can generally be classified into two categories: hyperkinetic movement disorders and hypokinetic movement disorders. Hyperkinetic movement disorders are caused by an increase in muscular activity and can cause abnormal and/or excessive movements, including tremors, dystonia, chorea, tics, myoclonus and stereotypes.

Hyperkinetic movement disorders often are often psychological in nature and arise through improper regulation of amine neurotransmitters in the basal ganglia.

A particular hyperkinetic movement disorder is Tourette's syndrome, which is an inherited neurological condition characterised by multiple physical and vocal tics. The tics are usually repetitive, but random, physical movements or vocal noises. The vocal tics can be of various forms and include repeating one's own words, the words of others or other sounds. Onset usually occurs in children and continues through to adolescence and adulthood.

While the tics associated with Tourette's syndrome are temporarily suppressible, those affected can usually only suppress their tics for limited time periods. There is yet to be an effective treatment to cover all types of tics in all patients, but certain medicaments for tic suppression have been developed.

It is known that dopamine receptor antagonists display an ability to suppress tics in Tourette's syndrome patients and a number of dopamine receptor antagonists are currently used in the suppression of Tourette's tics, such as fluphenazine, risperidone, haloperidol and pimozide.

Type 2 vesicular monoamine transporter (VMAT2) is a membrane protein responsible for the transportation of monoamine neurotransmitters, such as dopamine, serotonin and histamine, from cellular cytosol into synaptic vesicles. Inhibition of this protein hinders presynaptic neurons from releasing dopamine, resulting in a depletion of dopamine levels in the brain.

VMAT2 inhibitors can be used to treat the symptoms of Tourette's syndrome.

Tetrabenazine (Chemical name: 1,3,4,6,7,11b-hexahydro-9,10-dimethoxy-3-(2-methylpropyl)-2H-benzo(a)quinolizin-2-one) has been in use as a pharmaceutical drug since the late 1950s. Initially used as an anti-psychotic, tetrabenazine is currently used for treating hyperkinetic movement disorders such as Huntington's disease, hemiballismus, senile chorea, tic, tardive dyskinesia and Tourette's syndrome, see for example Jankovic et al., Am. J. Psychiatry. (1999) August; 156(8):1279-81 and Jankovic et al., Neurology (1997) February; 48(2):358-62.

The primary pharmacological action of tetrabenazine is to reduce the supply of monoamines (e.g. dopamine, serotonin, and norepinephrine) in the central nervous system by inhibiting the human vesicular monoamine transporter isoform 2 (hVMAT2). The drug also blocks post-synaptic dopamine receptors.

The central effects of tetrabenazine closely resemble those of reserpine, but it differs from reserpine in that it lacks activity at the VMAT1 transporter. The lack of activity at the VMAT1 transporter means that tetrabenazine has less peripheral activity than reserpine and consequently does not produce VMAT1-related side effects such as hypotension.

Tetrabenazine is an effective and safe drug for the treatment of a variety of hyperkinetic movement disorders and, in contrast to typical neuroleptics, has not been demonstrated to cause tardive dyskinesia. Nevertheless, tetrabenazine does exhibit a number of dose-related side effects including causing depression, parkinsonism, drowsiness, nervousness or anxiety, insomnia and, in rare cases, neuroleptic malignant syndrome, see for example the introductory section of WO2016/127133 (Neurocrine Biosciences).

The chemical structure of tetrabenazine is as shown below.

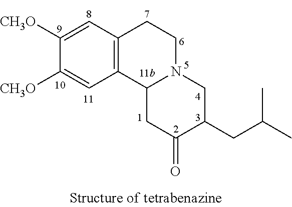

The compound has chiral centres at the 3 and 11 b carbon atoms and hence can, theoretically, exist in a total of four isomeric forms, as shown below.

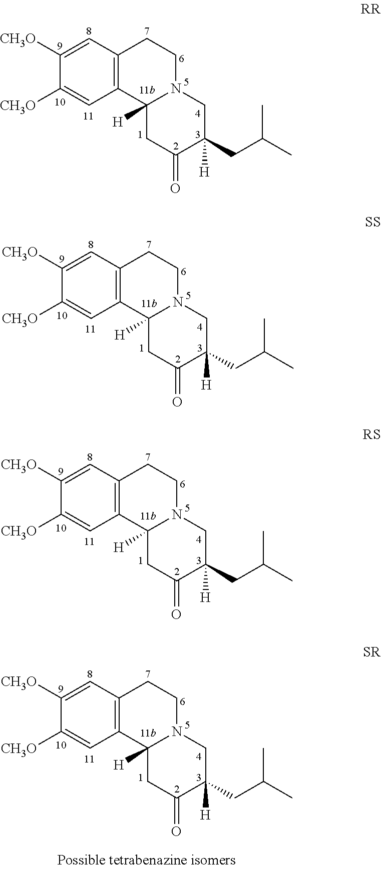

The stereochemistry of each isomer is defined using the “R and S” nomenclature developed by Cahn, Ingold and Prelog, see Advanced Organic Chemistry by Jerry March, 4th Edition, John Wiley & Sons, New York, 1992, pages 109-114. In this patent application, the designations “R” or “S” are given in the order of the position numbers of the carbon atoms. Thus, for example, RS is a shorthand notation for 3R,11bS. Similarly, when three chiral centres are present, as in the dihydrotetrabenazines described below, the designations “R” or “S” are listed in the order of the carbon atoms 2, 3 and 11 b. Thus, the 2R,3S,11bS isomer is referred to in short hand form as RSS and so on.

Commercially available tetrabenazine is a racemic mixture of the RR and SS isomers and it would appear that the RR and SS isomers are the most thermodynamically stable isomers.

Tetrabenazine has somewhat poor and variable bioavailability. It is extensively metabolised by first-pass metabolism, and little or no unchanged tetrabenazine is typically detected in the urine. It is known that at least some of the metabolites of tetrabenazine are dihydrotetrabenazines formed by reduction of the 2-keto group in tetrabenazine.

Dihydrotetrabenazine (Chemical name: 2-hydroxy-3-(2-methylpropyl)-1,3,4,6,7,11b-hexahydro-9,10-dimethoxy-benzo(a)quinolizine) has three chiral centres and can therefore exist in any of the following eight optical isomeric forms:

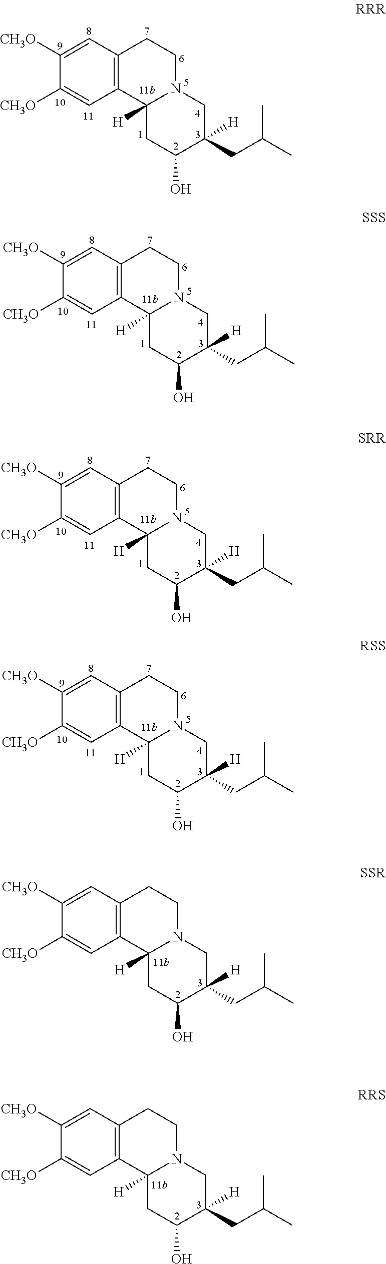

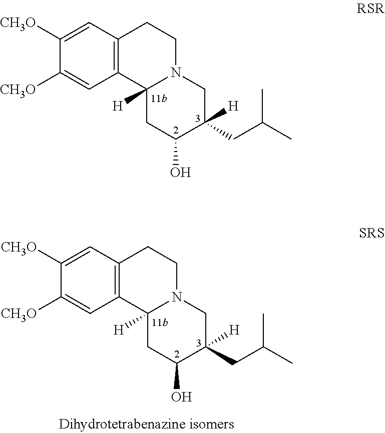

The synthesis and characterisation of all eight dihydrotetrabenazine isomers is described by Sun et al. (Eur. J. Med. Chem. (2011), 1841-1848).

Of the eight dihydrotetrabenazine isomers, four isomers are derived from the more stable RR and SS isomers of the parent tetrabenazine, namely the RRR, SSS, SRR and RSS isomers.

The RRR and SSS isomers are commonly referred to as “alpha (a)” dihydrotetrabenazines and can be referred to individually as (+)-α-dihydrotetrabenazine and (−)-α-dihydrotetrabenazine respectively. The alpha isomers are characterised by a trans relative orientation of the hydroxyl and 2-methylpropyl substituents at the 2- and 3-positions—see for example, Kilbourn et al., Chirality, 9:59-62 (1997) and Brossi et al., Helv. Chim. Acta., vol. XLI, No. 193, pp 1793-1806 (1958).

The SRR and RSS isomers are commonly referred to as “beta (β)” isomers and can be referred to individually as (+)-β-dihydrotetrabenazine and (−)-β-dihydrotetrabenazine respectively. The beta isomers are characterised by a cis relative orientation of the hydroxyl and 2-methylpropyl substituents at the 2- and 3-positions.

Although dihydrotetrabenazine is believed to be primarily responsible for the activity of the drug, there have been no studies published to date that contain evidence demonstrating which of the various stereoisomers of dihydrotetrabenazine is responsible for its biological activity. More specifically, there have been no published studies demonstrating which of the stereoisomers is responsible for the ability of tetrabenazine to treat movement disorders such as Tourette's syndrome.

Schwartz et al. (Biochem. Pharmacol. (1966), 15: 645-655) describes metabolic studies of tetrabenazine carried out in rabbits, dogs and humans. Schwartz et al. identified nine metabolites, five of which were unconjugated and the other four of which were conjugated with glucuronic acid. The five unconjugated metabolites were the alpha- and beta-dihydrotetrabenazines, their two oxidised analogues in which a hydroxyl group has been introduced into the 2-methylpropyl side chain, and oxidised tetrabenazine in which a hydroxyl group has been introduced into the 2-methylpropyl side chain. The four conjugated metabolites were all compounds in which the 9-methoxy group had been demethylated to give a 9-hydroxy compound. The chirality of the various metabolites was not studied and, in particular, there was no disclosure of the chirality of the individual α- and β-isomers.

Scherman et al., (Mol. Pharmacol. (1987), 33, 72-77 describes the stereospecificity of VMAT2 binding between racemic α- and β-dihydrotetrabenazine. They reported that α-dihydrotetrabenazine had a 3- to 4-fold higher affinity for the Chromaffin Granule Monoamine Transporter than the β-isomer, when studied in vitro. However, Scherman et al. does not disclose the resolution or testing of the individual enantiomers of the α- and β-dihydrotetrabenazines.

Mehvar et al. (J. Pharm. Sci. (1987), 76(6), 461-465) reported a study of the concentrations of tetrabenazine and dihydrotetrabenazine in the brains of rats following administration of either tetrabenazine or dihydrotetrabenazine. The study showed that despite its greater polarity, dihydrotetrabenazine was able to cross the blood-brain barrier. However, the stereochemistry of the dihydrotetrabenazine was not disclosed.

Mehvar et al. (Drug Metabolism and Disposition (1987), 15:2, 250-255) describes studies of the pharmacokinetics of tetrabenazine and dihydrotetrabenazine following administration of tetrabenazine to four patients affected by tardive dyskinesia. Oral administration of tetrabenazine resulted in low plasma concentrations of tetrabenazine but relatively high concentrations of dihydrotetrabenazine. However, the stereochemistry of the dihydrotetrabenazine formed in vivo was not reported.

Roberts et al. (Eur. J. Clin. Pharmacol. (1986), 29: 703-708) describes the pharmacokinetics of tetrabenazine and its hydroxy-metabolite in patients treated for involuntary movement disorders. Roberts et al. reported that tetrabenazine was extensively metabolised after oral administration resulting in very low plasma concentrations of tetrabenazine but much higher concentrations of a hydroxy-metabolite. Although they did not describe the identity of the hydroxy-metabolites, they suggested that the high plasma concentrations of the hydroxy-metabolites may be therapeutically important (since the metabolites were known to be pharmacologically active) and that, in view of the disclosure in Schwartz et al. (idem), the combination of cis and trans isomers (i.e. beta and alpha isomers) could be more therapeutically important than the parent drug.

Michael Kilbourn and collaborators at the University of Michigan Medical School have published a number of studies relating to the various isomers of dihydrotetrabenazines. Thus, in Med. Chem. Res. (1994), 5:113-126, Kilbourn et al. describe the use (+/−)-α-[11C]-dihydrotetrabenazine as in vivo imaging agents for VMAT2 binding studies.

In Eur. J. Pharmacol (1995) 278, 249-252, Kilbourn et al. reported competition binding studies using [³H]-tetrabenazine to study the in vitro binding affinity of (+)-, (−)-, and (+/−)-α-DHTBZ. The binding assays gave Ki values of 0.97 nM for (+)-α-dihydrotetrabenazine and 2.2 μM for (−)-α-dihydrotetrabenazine, thereby showing that the (+) alpha isomer has much greater binding affinity for the VMAT2 receptor than the (−) alpha isomer. However, no studies were reported, or conclusions drawn, as to the usefulness of either isomer in the treatment of movement disorders such as Tourette's syndrome.

In Chirality (1997) 9:59-62, Kilbourn et al. described studies aimed at identifying the absolute configuration of (+)-α-dihydrotetrabenazine from which they concluded that it has the 2R, 3R, 11bR configuration shown above. They also referred to the Schwartz et al. and Mehvar et al. articles discussed above as indicating that the α- and β-dihydrotetrabenazines are likely to be the pharmacologically active agents in the human brain but they drew no explicit conclusions as to the precise stereochemical identities of the active metabolites of tetrabenazine.

In Synapse (2002), 43:188-194, Kilbourn et al. described the use of (+)-α-[¹¹C]-dihydrotetrabenazine as an agent used to measure specific in vivo binding of the VMAT receptor, in “infusion to equilibrium methods”. They found that (−)-α-[¹¹C]-dihydrotetrabenazine produced a uniform brain distribution, consistent with the earlier observations that this enantiomer has a low VMAT affinity.

Sun et al. (idem) investigated the VMAT2 binding affinities of all eight dihydrotetrabenazine isomers. They found that all of the dextrorotatory enantiomers exhibited dramatically more potent VMAT2 binding activity than their corresponding laevorotatory enantiomers with the most active (+)-α-isomer being found to be the most active. However, Sun et al. did not carry out any investigations into the relative efficacies of the individual isomers in treating movement disorders such as Tourette's syndrome.

WO2015/120110 (Auspex) describes extended-release formulations that can contain any of a wide variety of different pharmacological agents, including tetrabenazine and dihydrotetrabenazine. WO2015/120110 discloses in general terms that the formulations may comprise between 5 mg and 30 mg of tetrabenazine or dihydrotetrabenazine and more specifically discloses extended-release formulations comprising tetrabenazine or dihydrotetrabenazine in amounts of 7.5 mg, 12.5 mg, 15 mg, 25 mg, 30 mg and 50 mg. However, there is no specific disclosure of formulations containing such amounts of (+)-α-dihydrotetrabenazine. Furthermore, there are no worked examples of any dihydrotetrabenazine formulations; but only formulations containing tetrabenazine.

WO 2006/053067 (Prestwick) describes the use of combinations of amantadine and a tetrabenazine compound (which can be tetrabenazine or dihydrotetrabenazine) for treating hyperkinetic movement disorders. WO 2006/053067 discloses that the amount of dihydrotetrabenazine administered may be 10-400 mg per day (although no examples are provided showing that the doses at the lower ends of these ranges are effective) and pharmaceutical compositions containing as little as 10 mg are also described. WO 2006/053067 does not specifically link any particular isomers of dihydrotetrabenazine to these amounts and, more particularly, does not disclose the specific use of 10 mg of (+)-α-dihydrotetrabenazine. No experimental data are provided in WO 2006/053067.

WO 2011/153157 (Auspex Pharmaceutical, Inc.) describes deuterated forms of dihydrotetrabenazine. Many deuterated forms of dihydrotetrabenazine are depicted but the application only provides sufficient information to allow a small number of the depicted compounds to be synthesised. Although racemic mixtures of d₆-α-dihydrotetrabenazine and d₆-β-dihydrotetrabenazine are disclosed, these mixtures were not resolved and the properties of the individual (+) and (−) isomers were not studied. Similarly, WO 2014/047167 (Auspex Pharmaceutical, Inc.) describes a number of deuterated forms of tetrabenazine and its derivatives. Again, the individual (+) and (−) isomers of deuterated forms of α- and β-dihydrotetrabenazine were not separated or studied.

It appears therefore that, up to the present, it remains unclear as to precisely which dihydrotetrabenazine isomers are responsible for the therapeutic properties resulting from the administration of tetrabenazine.

It has also remained somewhat unclear up until now whether (+)-α-dihydrotetrabenazine will provide a therapeutically useful effect in the treatment of movement disorders such as Tourette's syndrome without the accompaniment of unwanted side effects such as those described above. Thus, for example, whereas WO2016/127133 (Neurocrine Biosciences) refers to the Kilbourn et al. article in Chirality (idem) as indicating that (+)-α-dihydrotetrabenazine is the active metabolite of tetrabenazine. WO2016/127133, it also refers to the studies reported in Login et al. (1982), Ann. Neurology 12:257-62 and Reches et al., J. Pharmacol. Exp. Ther. (1983), 225:515-521 which indicate that tetrabenazine inhibits presynaptic and postsynaptic dopamine receptors in the rat brain. It is suggested in WO2016/127133 that this “off-target” activity of tetrabenazine may be responsible for some of the observed side effects of tetrabenazine.

As discussed above, it is known that tetrabenazine exhibits a number of dose-related side effects including causing depression and parkinsonism (see WO2016/127133). It appears that these side-effects may also be caused by VMAT2 inhibition and that consequently it is difficult to separate the therapeutic effect of tetrabenazine and tetrabenazine-derived compounds from these side-effects (see Müller, “Valbenazine granted breakthrough drug status for treating tardive dyskinesia”, Expert Opin. Investig. Drugs (2015), 24(6), pp. 737-742).

In an attempt to avoid or reduce the side-effects associated with tetrabenazine, a valine ester prodrug of (+)-α-dihydrotetrabenazine has been developed, known by its INN name, Valbenazine. The structure of Valbenazine is shown below:

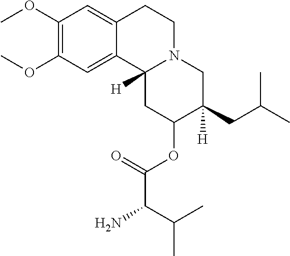 [SMILES: COc1cc2c(cc1OC)[C@H]1CC(OC(=O)[C@@H](N)C(C)C)[C@H](CC(C)C)CN1CC2]

As disclosed in U.S. Pat. No. 8,039,627, Valbenazine is prepared by reacting (+)-α-dihydrotetrabenazine with carbobenzyloxy-L-valine in dichloromethane and 4-dimethylaminopyridine (DMAP) in the presence of N,N′-dicyclohexylcarbodiimide (DCC) to give the intermediate 2-benzyloxycarbonylamino-3-methyl-butyric acid (2R,3R,1bR)-3-isobutyl-9,10-dimethoxy-1,3,4,6,7,11b-hexahydro-2H-pyrido[2,1-a]isoquinolin-2-yl ester. The intermediate is then hydrogenated over palladium on carbon to remove the benzyloxycarbonyl protecting group to give Valbenazine.

Müller (“Valbenazine granted breakthrough drug status for treating tardive dyskinesia”, Expert Opin. Investig. Drugs (2015), 24(6), pp. 737-742) describes a Phase IIb clinical study of Valbenazine (“KINECT 1”) in patients suffering from tardive dyskinesia. Although some reduction of symptoms was observed when doses of Valbenazine at 100 mg/day (equivalent to about 76 mg of (+)-α-dihydrotetrabenazine) were observed, subjects who received 50 mg/day of Valbenazine (equivalent to about 38 mg of (+)-α-dihydrotetrabenazine) did not show any significant signs of improvement, when scored with the abnormal involuntary movement scale (AIMS). Müller concluded that this study was more or less a failure, probably due to low Valbenazine dosing.

In a further study (“KINECT 2”) described in the same paper, subjects were initially dosed at 25 mg/day, with the dose range increasing to 75 mg/day. By the end of the study, when measurements were taken, 21 out of 34 of the subjects treated with Valbenazine were being dosed at 75 mg/day (O'Brien et al, “Kinect 2: NBI-98854 treatment of moderate to severe tardive dyskinesia” Mov. Disord. 2014; 29 (Suppl 1):829). The analysis does not provide a breakdown of the reduction in abnormal involuntary movements in patients who were being treated with 75 mg/day by the end of the trial and those who were being treated with 25 mg/day or 50 mg/day by the end of the trial.

A further Phase III trial of Valbenazine, reported by O'Brien et al (“KINECT 3 A randomised, Double-Blind Placebo-Controlled Phase 3 Trial of Valbenazine (NBI-98854) for Tardive Dyskinesia (PL02.003)”, Neurology (2016), 86(16), Supplement PL02.003) investigated the change in abnormal involuntary movements in Tardive Dyskinesia sufferers when administered with 40 mg or 80 mg of Valbenazine per day. It was found that 80 mg/day of Valbenazine resulted in a significant improvement in the Abnormal Involuntary Movement Score and it was concluded that 80 mg/day Valbenazine was associated with a significant improvement in Tardive Dyskinesia.

WO 2015/171802 (Neurocrine Biosciences, Inc.) describes methods for treating hyperkinetic diseases by administering therapeutic agents that produce plasma concentrations of (+)-α-dihydrotetrabenazine such that there is a Cₘₐₓ of between about 15 ng/ml and 60 ng/ml and a Cₘᵢₙ of at least 15 ng/ml over an eight hour period. Although it is suggested in WO 2015/171802 that this can be accomplished by administering (+)-α-dihydrotetrabenazine per se, the experiments described in WO 2015/171802 only provide data for (+)-α-dihydrotetrabenazine levels achieved after the administration of Valbenazine. In Example 1 of WO 2015/171802, it is concluded that a concentration of 30 ng/ml of (+)-α-dihydrotetrabenazine in plasma is an appropriate target and that exposures below 15 ng/ml are suboptimal across the general tardive dyskinesia (TD) population. In Example 2 of WO 2015/171802, it is disclosed that a 50 mg dose of Valbenazine appeared to maintain the required plasma levels of (+)-α-dihydrotetrabenazine.

WO2016/210180 (Neurocrine Biosciences) discloses the use of VMAT2 inhibitors for treating various neurological disorders. (+)-α-dihydrotetrabenazine is mentioned as an example of a VMAT2 inhibitor and therapeutic agents that provide plasma concentrations similar to those disclosed in WO2015/171802 are disclosed. The VMAT2 inhibitory compounds specifically exemplified in WO2016/210180 are Valbenazine and [(2R,3S,11bR)-9,10-dimethoxy-3-(2-methylpropyl)-1H,2H,3H,4H,6H,7H,11bH-pyrido[2,1-a]isoquinolin-2-yl]methanol.

### The Invention

Investigations made by the present applicant indicate that (+)-α-dihydrotetrabenazine per se having the chemical name, “(R,R,R)-3-isobutyl-9,10-dimethyloxy-1,3,4,5,7,11b-hexahydro-2H-pyrido[2,1-a]isoquinolin-2-ol”, and having the formula (I)

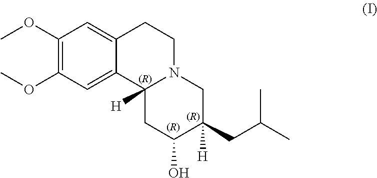

is effective in the treatment of movement disorders at much lower doses than could have been predicted from the literature (for example from WO 2015/171802) and that its use at such lower doses can avoid or minimize the unwanted side effects associated with tetrabenazine.

More particularly, experiments carried out by the present inventors indicate that movement disorders such as Tourette's syndrome can be treated effectively by administering much lower doses of (+)-α-dihydrotetrabenazine per se than the doses of Valbenazine required in WO 2015/171802e.

In addition, contrary to the teachings of WO 2015/171802, it has been found that by administering (+)-α-dihydrotetrabenazine at a dose to provide blood plasma concentrations that do not exceed 15 ng/mL for a period of 8 hours, efficacious results are achieved.

The use of lower dosages is expected to reduce the biological side-effects associated with tetrabenazine and tetrabenazine derivatives, including parkinsonism and depression.

An advantage of using (+)-α-dihydrotetrabenazine per se rather than the prodrug Valbenazine is that it avoids the two additional synthetic steps and additional purification step required to prepare Valbenazine from (+)-α-dihydrotetrabenazine. In addition, the release of the active (+)-α-dihydrotetrabenazine into the blood plasma is not limited by the rate of degradation of the prodrug.

A further unexpected benefit of using low doses of (+)-α-dihydrotetrabenazine is that experiments conducted by the present inventors suggest that whereas abnormal movements of the type found in movement disorders will be reduced or suppressed by the drug, normal movements will not be. This is in contrast to risperidone, a well-used treatment for movement disorders, where the levels of both normal and abnormal movements can be reduced by administration of the drug.

On the basis of these findings, it is envisaged that low doses of (+)-α-dihydrotetrabenazine will be useful in the prophylaxis or treatment of inter alia the disease states and conditions for which tetrabenazine is currently used or proposed. Thus, by way of example, and without limitation, it is envisaged that low doses of (+)-α-dihydrotetrabenazine may be used for the treatment of hyperkinetic movement disorders such as Huntington's disease, hemiballismus, senile chorea, tic disorders, tardive dyskinesia, dystonia and, in particular, Tourette's syndrome.

In accordance with the invention, a subject can initially be administered a very low dose (for example from 0.5 mg to 5 mg) of (+)-α-dihydrotetrabenazine or pharmaceutically acceptable salt thereof, and the therapeutic effect of the initial dose evaluated before, where necessary and desirable, increasing the dose. Thus, starting from a very low initial dose, a subject can be titrated to identify the optimum dosage for a particular movement disorder. Such low initial doses are far below the lowest dosages of tetrabenazine currently administered in clinical practice.

Accordingly, in a first aspect (Embodiment 1.1), the invention provides a method of treating a movement disorder in a subject in need thereof, which treatment comprises the steps of:

(a) administering to the subject an initial daily dosage of (+)-α-dihydrotetrabenazine or pharmaceutically acceptable salt thereof, wherein the initial daily dosage is an amount of (+)-α-dihydrotetrabenazine or pharmaceutically acceptable salt thereof corresponding to from 0.5 mg to 5 mg of (+)-α-dihydrotetrabenazine free base;
(b) carrying out a clinical evaluation of the subject for efficacy and side effects arising from the treatment;
(c) where the clinical evaluation (b) has established that an increased daily dosage of (+)-α-dihydrotetrabenazine or pharmaceutically acceptable salt thereof is desirable, administering an increased daily dosage which is greater than the initial daily dosage by an incremental amount of (+)-α-dihydrotetrabenazine or pharmaceutically acceptable salt thereof corresponding to from 0.5 mg to 5 mg of (+)-α-dihydrotetrabenazine free base; or, where the clinical evaluation has established that an increased daily dosage is not desirable, either maintaining the initial daily dosage, reducing the dosage, or discontinuing the treatment;
(d) where an increased daily dosage has been administered, carrying out a further clinical evaluation of the subject for efficacy and side effects arising from the treatment with the increased daily dosage;
(e) where the further clinical evaluation (d) has established that a further increased daily dosage of (+)-α-dihydrotetrabenazine or pharmaceutically acceptable salt thereof is desirable, administering a further increased daily dosage which is greater than an immediately preceding daily dosage by an incremental amount of (+)-α-dihydrotetrabenazine or pharmaceutically acceptable salt thereof corresponding to from 0.5 mg to 5 mg of (+)-α-dihydrotetrabenazine free base; or, where the clinical evaluation has established that a further increased daily dosage is not desirable, maintaining the immediately preceding daily dosage, reducing the immediately preceding dosage or discontinuing the treatment; and
(f) optionally repeating steps (d) and (e) as often as desired until an optimum daily dosage is reached.

In particular embodiments of the aforementioned method, there are provided:

1.2 A method according to Embodiment 1.1 wherein the initial daily dosage of (+)-α-dihydrotetrabenazine or pharmaceutically acceptable salt thereof, is an amount corresponding to from 0.5 mg to 3 mg of (+)-α-dihydrotetrabenazine free base.
1.3 A method according to Embodiment 1.2 wherein the initial daily dosage of (+)-α-dihydrotetrabenazine or pharmaceutically acceptable salt thereof, is an amount corresponding to from 0.5 mg to 2 mg of (+)-α-dihydrotetrabenazine free base.
1.4 A method according to Embodiment 1.2 wherein the initial daily dosage of (+)-α-dihydrotetrabenazine or pharmaceutically acceptable salt thereof, is an amount corresponding to 0.5 mg, 1 mg, 1.5 mg, or 2 mg of (+)-α-dihydrotetrabenazine free base.
1.5 A method according to Embodiment 1.4 wherein the initial daily dosage of (+)-α-dihydrotetrabenazine or pharmaceutically acceptable salt thereof, is an amount corresponding to 0.5 mg of (+)-α-dihydrotetrabenazine free base.
1.6 A method according to Embodiment 1.4 wherein the initial daily dosage of (+)-α-dihydrotetrabenazine or pharmaceutically acceptable salt thereof, is an amount corresponding to 1 mg of (+)-α-dihydrotetrabenazine free base.
1.7 A method according to Embodiment 1.4 wherein the initial daily dosage of (+)-α-dihydrotetrabenazine or pharmaceutically acceptable salt thereof, is an amount corresponding to 1.5 mg of (+)-α-dihydrotetrabenazine free base.
1.8 A method according to Embodiment 1.4 wherein the initial daily dosage of (+)-α-dihydrotetrabenazine or pharmaceutically acceptable salt thereof, is an amount corresponding to 2 mg of (+)-α-dihydrotetrabenazine free base.
1.9 A method according to any one of Embodiments 1.1 to 1.8 wherein the increased daily dosage in step (c) is an amount which is greater than the initial daily dosage by an incremental amount of (+)-α-dihydrotetrabenazine or pharmaceutically acceptable salt thereof corresponding to from 0.5 mg to 3 mg of (+)-α-dihydrotetrabenazine free base.
1.10 A method according to Embodiment 1.9 wherein the increased daily dosage in step (c) is an amount which is greater than the initial daily dosage by an incremental amount of (+)-α-dihydrotetrabenazine or pharmaceutically acceptable salt thereof corresponding to from 0.5 mg to 2 mg of (+)-α-dihydrotetrabenazine free base.
1.11 A method according to Embodiment 1.10 wherein the increased daily dosage in step (c) is an amount which is greater than the initial daily dosage by an incremental amount of (+)-α-dihydrotetrabenazine or pharmaceutically acceptable salt thereof corresponding to 0.5 mg, 1 mg, 1.5 mg, or 2 mg of (+)-α-dihydrotetrabenazine free base.
1.12 A method according to Embodiment 1.11 wherein the increased daily dosage in step (c) is an amount which is greater than the initial daily dosage by an incremental amount of (+)-α-dihydrotetrabenazine or pharmaceutically acceptable salt thereof corresponding to 0.5 mg of (+)-α-dihydrotetrabenazine free base.
1.13 A method according to Embodiment 1.11 wherein the increased daily dosage in step (c) is an amount which is greater than the initial daily dosage by an incremental amount of (+)-α-dihydrotetrabenazine or pharmaceutically acceptable salt thereof corresponding to 1 mg of (+)-α-dihydrotetrabenazine free base.
1.14 A method according to Embodiment 1.11 wherein the increased daily dosage in step (c) is an amount which is greater than the initial daily dosage by an incremental amount of (+)-α-dihydrotetrabenazine or pharmaceutically acceptable salt thereof corresponding to 1.5 mg of (+)-α-dihydrotetrabenazine free base.
1.15 A method according to Embodiment 1.11 wherein the increased daily dosage in step (c) is an amount which is greater than the initial daily dosage by an incremental amount of (+)-α-dihydrotetrabenazine or pharmaceutically acceptable salt thereof corresponding to 2 mg of (+)-α-dihydrotetrabenazine free base.
1.16 A method according to any one of Embodiments 1.1 to 1.15 wherein the further increased daily dosage in step (e) is greater than an immediately preceding daily dosage by an incremental amount of (+)-α-dihydrotetrabenazine or pharmaceutically acceptable salt thereof corresponding to from 0.5 mg to 3 mg of (+)-α-dihydrotetrabenazine free base.
1.17 A method according to Embodiment 1.16 wherein the further increased daily dosage in step (e) is greater than an immediately preceding daily dosage by an incremental amount of (+)-α-dihydrotetrabenazine or pharmaceutically acceptable salt thereof corresponding to from 0.5 mg to 2 mg of (+)-α-dihydrotetrabenazine free base.
1.18 A method according to Embodiment 1.17 wherein the further increased daily dosage in step (e) is greater than an immediately preceding daily dosage by an incremental amount of (+)-α-dihydrotetrabenazine or pharmaceutically acceptable salt thereof corresponding to 0.5 mg, 1 mg, 1.5 mg, or 2 mg of (+)-α-dihydrotetrabenazine free base.
1.19 A method according to Embodiment 1.18 wherein the further increased daily dosage in step (e) is greater than an immediately preceding daily dosage by an incremental amount of (+)-α-dihydrotetrabenazine or pharmaceutically acceptable salt thereof corresponding to 0.5 mg of (+)-α-dihydrotetrabenazine free base.
1.20 A method according to Embodiment 1.18 wherein the further increased daily dosage in step (e) is greater than an immediately preceding daily dosage by an incremental amount of (+)-α-dihydrotetrabenazine or pharmaceutically acceptable salt thereof corresponding to 1 mg of (+)-α-dihydrotetrabenazine free base.
1.21 A method according to Embodiment 1.18 wherein the further increased daily dosage in step (e) is greater than an immediately preceding daily dosage by an incremental amount of (+)-α-dihydrotetrabenazine or pharmaceutically acceptable salt thereof corresponding to 1.5 mg of (+)-α-dihydrotetrabenazine free base.
1.22 A method according to Embodiment 1.18 wherein the further increased daily dosage in step (e) is greater than an immediately preceding daily dosage by an incremental amount of (+)-α-dihydrotetrabenazine or pharmaceutically acceptable salt thereof corresponding to 2 mg of (+)-α-dihydrotetrabenazine free base.
1.23 A method according to any one of Embodiments 1.1 to 1.22 wherein the treatment comprises the administration of a maximum (e.g. optimized) daily dosage of (+)-α-dihydrotetrabenazine or pharmaceutically acceptable salt thereof, which is an amount corresponding to no greater than 20 mg of (+)-α-dihydrotetrabenazine free base.
1.24 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 17.5 mg of (+)-α-dihydrotetrabenazine free base.
1.25 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 15 mg of (+)-α-dihydrotetrabenazine free base.
1.26 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 12.5 mg of (+)-α-dihydrotetrabenazine free base.
1.27 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 10 mg of (+)-α-dihydrotetrabenazine free base.
1.28 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 9 mg of (+)-α-dihydrotetrabenazine free base.
1.29 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 8 mg of (+)-α-dihydrotetrabenazine free base.
1.30 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 7.5 mg of (+)-α-dihydrotetrabenazine free base.
1.31 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 7 mg of (+)-α-dihydrotetrabenazine free base.
1.32 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 6 mg of (+)-α-dihydrotetrabenazine free base.
1.33 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 5 mg of (+)-α-dihydrotetrabenazine free base.
1.34 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 4 mg of (+)-α-dihydrotetrabenazine free base.
1.35 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 3 mg of (+)-α-dihydrotetrabenazine free base.
1.36 A method according to Embodiment 1.23 wherein the treatment comprises the administration of a maximum (e.g. optimized) daily dosage of (+)-α-dihydrotetrabenazine or pharmaceutically acceptable salt thereof, which is an amount corresponding to no greater than 2.5 mg of (+)-α-dihydrotetrabenazine free base.

The amount of (+)-α-dihydrotetrabenazine or a pharmaceutically acceptable salt thereof to be administered to a subject can be determined, at least initially, by first determining the weight of the subject and then administering a dose appropriate for the subject's weight. Thus, in further embodiments, the invention provides:

1.37 A method according to any one of Embodiments 1.1 to 1.23 wherein the treatment comprises determining an approximate weight of the subject and:
(i) when the subject has a weight of 30 kg to 50 kg, administering a maximum (e.g. optimized) daily amount of (+)-α-dihydrotetrabenazine or a pharmaceutically acceptable salt thereof corresponding to from 2 mg to 7.5 mg of (+)-α-dihydrotetrabenazine free base;
(ii) when the subject has a weight of 50 kg to 75 kg, administering a maximum (e.g. optimized) daily amount of (+)-α-dihydrotetrabenazine or a pharmaceutically acceptable salt thereof corresponding to from 5 mg to 10 mg of (+)-α-dihydrotetrabenazine free base;
(iii) when the subject has a weight of 75 kg to 95 kg, administering a maximum (e.g. optimized) daily amount of (+)-α-dihydrotetrabenazine or a pharmaceutically acceptable salt thereof corresponding to from 7.5 mg to 15 mg of (+)-α-dihydrotetrabenazine free base; or
(iv) when the subject has a weight of greater than 95 kg, administering a maximum (e.g. optimized) daily amount of (+)-α-dihydrotetrabenazine or a pharmaceutically acceptable salt thereof corresponding to from 15 mg to 20 mg of (+)-α-dihydrotetrabenazine free base the amount of (+)-α-dihydrotetrabenazine administered per day is from 15 mg to 20 mg.
1.38 A method of treating a movement disorder in a subject in need thereof, which treatment comprises administering to the subject an effective amount of (+)-α-dihydrotetrabenazine or a pharmaceutically acceptable salt thereof wherein:
(i) when the subject has a weight of 30 kg to 50 kg, the said effective amount is a daily amount of (+)-α-dihydrotetrabenazine or a pharmaceutically acceptable salt thereof corresponding to from 2 mg to 7.5 mg of (+)-α-dihydrotetrabenazine free base;
(ii) when the subject has a weight of 50 kg to 75 kg, the said effective amount is a daily amount of (+)-α-dihydrotetrabenazine or a pharmaceutically acceptable salt thereof corresponding to from 5 mg to 10 mg of (+)-α-dihydrotetrabenazine free base;
(iii) when the subject has a weight of 75 kg to 95 kg, the said effective amount is a daily amount of (+)-α-dihydrotetrabenazine or a pharmaceutically acceptable salt thereof corresponding to from 7.5 mg to 15 mg of (+)-α-dihydrotetrabenazine free base; or
(iv) when the subject has a weight of greater than 95 kg, the said effective amount is a daily amount of (+)-α-dihydrotetrabenazine or a pharmaceutically acceptable salt thereof corresponding to from 15 mg to 20 mg of (+)-α-dihydrotetrabenazine free base the amount of (+)-α-dihydrotetrabenazine administered per day is from 15 mg to 20 mg.
1.39 A method of treating a movement disorder in a subject in need thereof, which treatment comprises administering to the subject an effective amount of (+)-α-dihydrotetrabenazine or a pharmaceutically acceptable salt thereof, wherein the said effective amount corresponds to an amount of (+)-α-dihydrotetrabenazine free base of from 0.05 mg/kg to 0.3 mg/kg per day.
1.40 A method according to Embodiment 1.39 wherein the said effective amount corresponds to an amount of (+)-α-dihydrotetrabenazine free base of from 0.1 mg/kg to 0.2 mg/kg per day.

The amount of (+)-α-dihydrotetrabenazine or a pharmaceutically acceptable salt thereof administered may be determined by the blood plasma concentrations of (+)-α-dihydrotetrabenazine that it is desired to achieve in the subject or a desired level of blocking of the VMAT2 proteins. Accordingly, the invention further provides:

1.41 A method of treatment of a movement disorder in a subject in need thereof wherein the treatment comprises administering to a subject a therapeutically effective amount of (+)-α-dihydrotetrabenazine or a pharmaceutically acceptable salt thereof, wherein the said effective amount is sufficient to achieve an average blood plasma C_{avg} concentration of (+)-α-dihydrotetrabenazine, when measured over a period of three hours, in the range from 3 ng/ml to 15 ng/ml.
1.42 A method according to any one of Embodiments 1.1 to 1.40 wherein the daily dosage, daily amount or effective amount of (+)-α-dihydrotetrabenazine or a pharmaceutically acceptable salt thereof administered to the subject is sufficient to cause a level of blocking of between 50% and 85% of VMAT2 proteins in the subject.
1.43 A method according to any one of Embodiments 1.11 to 40 wherein the daily dosage, daily amount or effective amount of (+)-α-dihydrotetrabenazine or a pharmaceutically acceptable salt thereof administered to the subject is sufficient to cause a level of blocking of between 55% and 85% of VMAT2 proteins in the brain of the subject.
1.44 A method according to Embodiment 1.42 or Embodiment 1.43 which treatment comprises administering to the subject an amount of (+)-α-dihydrotetrabenazine or pharmaceutically acceptable salt thereof sufficient to cause a level of blocking of between 70% and 85% of the VMAT2 proteins in the subject or, as the case may be, in the brain of the subject.

In further Embodiments, the invention provides:

1.45 A method according to any one of Embodiments 1.1 to 1.44 wherein the (+)-α-dihydrotetrabenazine or pharmaceutically acceptable salt thereof is administered to the subject once per day.
1.46 A method according to any one of Embodiments 1.1 to 1.44 wherein the (+)-α-dihydrotetrabenazine or pharmaceutically acceptable salt thereof is administered to the subject twice per day.
1.47 A method according to any one of Embodiments 1.1 to 1.46 wherein the (+)-α-dihydrotetrabenazine or pharmaceutically acceptable salt thereof is not administered in combination with a therapeutically effective amount of amantadine.
1.48. A method according to Embodiment 1.47 wherein the (+)-α-dihydrotetrabenazine or pharmaceutically acceptable salt thereof is not administered in combination with any amount of amantadine.
1.49 A method according to any one of Embodiments 1.1 to 1.48 wherein the (+)-α-dihydrotetrabenazine or pharmaceutically acceptable salt thereof is administered as a non-extended or delayed release dosage form (e.g. an immediate release unit dosage form).
1.50 A method according to any one of Embodiments 1.1 to 1.49 wherein the (+)-α-dihydrotetrabenazine or pharmaceutically acceptable salt is administered orally.
1.51 A method according to Embodiment 1.50 wherein the (+)-α-dihydrotetrabenazine or pharmaceutically acceptable salt is administered in the form of a tablet, capsule, solution, syrup or suspension.
1.51A A method according to Embodiment 1.50 wherein the (+)-α-dihydrotetrabenazine or pharmaceutically acceptable salt is administered in the form of a tablet.
1.51B A method according to Embodiment 1.50 wherein the (+)-α-dihydrotetrabenazine or pharmaceutically acceptable salt is administered in the form of a solution.
1.51C A method according to Embodiment 1.50 wherein the (+)-α-dihydrotetrabenazine or pharmaceutically acceptable salt is administered in the form of a syrup.
1.51D A method according to Embodiment 1.50 wherein the (+)-α-dihydrotetrabenazine or pharmaceutically acceptable salt is administered in the form of a suspension.
1.52 A method according to Embodiment 1.50 wherein the (+)-α-dihydrotetrabenazine or pharmaceutically acceptable salt is administered in the form of a capsule.

The invention also provides novel low dose unit dosage forms containing (+)-α-dihydrotetrabenazine or pharmaceutically acceptable salt thereof. Accordingly, in further embodiments, the invention provides:

1.53 A unit dosage form comprising between 0.5 mg and 20 mg of (+)-α-dihydrotetrabenazine, or a pharmaceutically acceptable salt thereof, and a pharmaceutically acceptable excipient.
1.54 A unit dosage form according to Embodiment 1.53 which is formulated for oral administration.
1.55 A unit dosage form according to Embodiment 1.54 which is a capsule or tablet.
1.56. A unit dosage form according to any one of Embodiments 1.53 to 1.55 comprising between 1 mg and 20 mg of (+)-α-dihydrotetrabenazine, or a pharmaceutically acceptable salt thereof, and a pharmaceutically acceptable excipient.
1.57 A unit dosage form according to any one of Embodiments 1.53 to 1.55 comprising between 2 mg and 20 mg of (+)-α-dihydrotetrabenazine, or a pharmaceutically acceptable salt thereof, and a pharmaceutically acceptable excipient.
1.58 A unit dosage form according to any one of Embodiments 1.53 to 1.55 comprising between 0.5 mg and 10 mg of (+)-α-dihydrotetrabenazine, or a pharmaceutically acceptable salt thereof, and a pharmaceutically acceptable excipient.
1.59 A unit dosage form according to any one of Embodiments 1.53 to 1.55 comprising between 0.5 mg and 7.5 mg of (+)-α-dihydrotetrabenazine, or a pharmaceutically acceptable salt thereof, and a pharmaceutically acceptable excipient.
1.60 A unit dosage form according to any one of Embodiments 1.53 to 1.55 comprising between 1 mg and 10 mg of (+)-α-dihydrotetrabenazine, or a pharmaceutically acceptable salt thereof, and a pharmaceutically acceptable excipient.
1.61 A unit dosage form according to any one of Embodiments 1.53 to 1.55 comprising between 1 mg and 7.5 mg of (+)-α-dihydrotetrabenazine, or a pharmaceutically acceptable salt thereof, and a pharmaceutically acceptable excipient.
1.62 A unit dosage form according to any one of Embodiments 1.53 to 1.55 comprising between 3 mg and 20 mg of (+)-α-dihydrotetrabenazine, or a pharmaceutically acceptable salt thereof, and a pharmaceutically acceptable excipient.
1.63 A unit dosage form according to any one of Embodiments 1.53 to 1.55 comprising between 2 mg and 15 mg of (+)-α-dihydrotetrabenazine, or a pharmaceutically acceptable salt thereof, and a pharmaceutically acceptable excipient.
1.64 A unit dosage form according to any one of Embodiments 1.53 to 1.55 comprising between 3 mg and 15 mg of (+)-α-dihydrotetrabenazine, or a pharmaceutically acceptable salt thereof, and a pharmaceutically acceptable excipient.
1.65 A unit dosage form according to any one of Embodiments 1.53 to 1.55 comprising between 4 mg and 15 mg of (+)-α-dihydrotetrabenazine, or a pharmaceutically acceptable salt thereof, and a pharmaceutically acceptable excipient.
1.66 A unit dosage form according to any one of Embodiments 1.53 to 1.55 comprising between 5 mg and 15 mg of (+)-α-dihydrotetrabenazine, or a pharmaceutically acceptable salt thereof, and a pharmaceutically acceptable excipient.
1.67 A unit dosage form according to any one of Embodiments 1.53 to 1.55 comprising approximately 0.5 mg of (+)-α-dihydrotetrabenazine, or a pharmaceutically acceptable salt thereof, and a pharmaceutically acceptable excipient.
1.68 A unit dosage form according to any one of Embodiments 1.53 to 1.55 comprising approximately 1 mg of (+)-α-dihydrotetrabenazine, or a pharmaceutically acceptable salt thereof, and a pharmaceutically acceptable excipient.
1.69 A unit dosage form according to any one of Embodiments 1.53 to 1.55 comprising approximately 1.5 mg of (+)-α-dihydrotetrabenazine, or a pharmaceutically acceptable salt thereof, and a pharmaceutically acceptable excipient.
1.70 A unit dosage form according to any one of Embodiments 1.53 to 1.55 comprising approximately 2 mg of (+)-α-dihydrotetrabenazine, or a pharmaceutically acceptable salt thereof, and a pharmaceutically acceptable excipient.
1.71 A unit dosage form according to any one of Embodiments 1.53 to 1.55 comprising approximately 2.5 mg of (+)-α-dihydrotetrabenazine, or a pharmaceutically acceptable salt thereof, and a pharmaceutically acceptable excipient.
1.72 A unit dosage form according to any one of Embodiments 1.53 to 1.55 comprising approximately 3 mg of (+)-α-dihydrotetrabenazine, or a pharmaceutically acceptable salt thereof, and a pharmaceutically acceptable excipient.
1.73 A unit dosage form according to any one of Embodiments 1.53 to 1.55 comprising approximately 3.5 mg of (+)-α-dihydrotetrabenazine, or a pharmaceutically acceptable salt thereof, and a pharmaceutically acceptable excipient.
1.74 A unit dosage form according to any one of Embodiments 1.53 to 1.55 comprising approximately 4 mg of (+)-α-dihydrotetrabenazine, or a pharmaceutically acceptable salt thereof, and a pharmaceutically acceptable excipient.
1.75 A unit dosage form according to any one of Embodiments 1.53 to 1.55 comprising approximately 4.5 mg of (+)-α-dihydrotetrabenazine, or a pharmaceutically acceptable salt thereof, and a pharmaceutically acceptable excipient.
1.76 A unit dosage form according to any one of Embodiments 1.53 to 1.55 comprising approximately 5 mg of (+)-α-dihydrotetrabenazine, or a pharmaceutically acceptable salt thereof, and a pharmaceutically acceptable excipient.
1.77 A unit dosage form according to any one of Embodiments 1.53 to 1.55 comprising approximately 7.5 mg of (+)-α-dihydrotetrabenazine, or a pharmaceutically acceptable salt thereof, and a pharmaceutically acceptable excipient.
1.78 A unit dosage form according to any one of Embodiments 1.53 to 1.55 comprising approximately 10 mg of (+)-α-dihydrotetrabenazine, or a pharmaceutically acceptable salt thereof, and a pharmaceutically acceptable excipient.
1.79 A unit dosage form according to any one of Embodiments 1.53 to 1.55 comprising approximately 12.5 mg of (+)-α-dihydrotetrabenazine, or a pharmaceutically acceptable salt thereof, and a pharmaceutically acceptable excipient.
1.80 A unit dosage form according to any one of Embodiments 1.53 to 1.55 comprising approximately 15 mg of (+)-α-dihydrotetrabenazine, or a pharmaceutically acceptable salt thereof, and a pharmaceutically acceptable excipient.
1.81 A unit dosage form according to any one of Embodiments 1.53 to 1.55 comprising from 0.5 mg to 3 mg of (+)-α-dihydrotetrabenazine, or a pharmaceutically acceptable salt thereof, and a pharmaceutically acceptable excipient.
1.82 A unit dosage form according to any one of Embodiments 1.53 to 1.55 comprising from 0.5 mg to 2 mg of (+)-α-dihydrotetrabenazine, or a pharmaceutically acceptable salt thereof, and a pharmaceutically acceptable excipient.
1.83 A unit dosage form according to any one of Embodiments 1.53 to 1.55 comprising from 1 mg to 3 mg of (+)-α-dihydrotetrabenazine, or a pharmaceutically acceptable salt thereof, and a pharmaceutically acceptable excipient.
1.84 A method according to any one of Embodiments 1.1 to 1.52 wherein the (+)-α-dihydrotetrabenazine, or a pharmaceutically acceptable salt thereof, is administered as a unit dosage form according to any one of Embodiments 1.53 to 1.83.

The methods and unit dosage forms defined and described above are typically for use in the treatment of a hyperkinetic movement disorder such as Huntington's disease, hemiballismus, senile chorea, tic disorders, tardive dyskinesia, dystonia, myoclonus and Tourette's syndrome.

More particularly, the unit dosage forms described above are for use in the treatment of a hyperkinetic movement disorder selected from tic disorders, tardive dyskinesia and Tourette's syndrome.

In one particular embodiment, the unit dosage forms described above are for use in the treatment of tardive dyskinesia.

In another particular embodiment, the unit dosage forms described above are for use in the treatment of Tourette's syndrome.

The term “treatment” as used herein in the context of treating a condition or disorder, pertains generally to treatment and therapy in which some desired therapeutic effect is achieved, for example, the inhibition of the progress of the condition, and includes a reduction in the rate of progress, a halt in the rate of progress, amelioration of the condition, diminishment or alleviation of at least one symptom associated or caused by the condition being treated and cure of the condition. When the hyperkinetic movement disorder being treated is Tourette's Syndrome, treatment of the disorder may pertain to a reduction of the incidence or severity of tics.

Accordingly, in further embodiments, the invention also; provides:

1.85 A method according to any one of Embodiments 1.1 to 1.52 and 1.84 wherein the movement disorder is selected from tardive dyskinesia, Tourette's syndrome and Huntington's disease.
1.86 A method according to Embodiment 1.85 wherein the movement disorder is Tourette's Syndrome.
1.87 A method according to Embodiment 1.85 wherein the movement disorder is Huntington's Disease.
1.88 A method according to Embodiment 1.85 wherein the movement disorder is tardive dyskinesia.

As described above, the present inventors have found that low dosage regimes of (+)-α-dihydrotetrabenazine are useful in blocking the VMAT2 receptor in the treatment of movement disorders. Accordingly, in further embodiments, the invention provides:

1.89 (+)-α-dihydrotetrabenazine for use in a method for the treatment of a movement disorder, wherein the treatment comprises administering to a subject an amount of (+)-α-dihydrotetrabenzine between 0.5 mg and 20 mg per day.
1.90 A method of treatment of a movement disorder in a subject in need thereof (e.g. a mammalian subject such as a human), which treatment comprises administering to the subject an amount of (+)-α-dihydrotetrabenazine between 0.5 mg and 20 mg per day.
1.91 The use of (+)-α-dihydrotetrabenazine for the manufacture of a medicament for the treatment of a movement disorder, which treatment comprises administering to the subject an amount of (+)-α-dihydrotetrabenazine between 0.5 mg and 20 mg per day.

In further embodiments, there is provided

1.92 (+)-α-dihydrotetrabenazine for use, a method or a use as described herein, wherein the treatment comprises administering to the subject an amount of (+)-α-dihydrotetrabenazine between 1 mg and 20 mg per day.
1.93 (+)-α-dihydrotetrabenazine for use, a method or a use as described herein, wherein the treatment comprises administering to the subject an amount of (+)-α-dihydrotetrabenazine between 2 mg and 20 mg per day.
1.94 (+)-α-dihydrotetrabenazine for use, a method or a use as described herein, wherein the treatment comprises administering to the subject an amount of (+)-α-dihydrotetrabenazine 3 mg and 20 mg per day.
1.95 (+)-α-dihydrotetrabenazine for use, a method or a use as described herein, wherein the treatment comprises administering to the subject an amount of (+)-α-dihydrotetrabenazine 2 mg and 15 mg per day.
1.96 (+)-α-dihydrotetrabenazine for use, a method or a use as described herein, wherein the treatment comprises administering to the subject an amount of (+)-α-dihydrotetrabenazine between 3 mg and 15 mg per day.
1.97 (+)-α-dihydrotetrabenazine for use, a method or a use as described herein, wherein the treatment comprises administering to the subject an amount of (+)-α-dihydrotetrabenazine between 5 mg and 15 mg per day.
1.98 (+)-α-dihydrotetrabenazine for use, a method or a use as described herein, wherein the treatment comprises administering to the subject an amount of (+)-α-dihydrotetrabenazine between 5 mg and 10 mg per day.
1.99 (+)-α-dihydrotetrabenazine for use, a method or a use as described herein, wherein the treatment comprises administering to the subject an amount of (+)-α-dihydrotetrabenazine of approximately 5 mg per day.
1.100 (+)-α-dihydrotetrabenazine for use, a method or a use as described herein, wherein the treatment comprises administering to the subject an amount of (+)-α-dihydrotetrabenazine of approximately 7.5 mg per day.
1.101 (+)-α-dihydrotetrabenazine for use, a method or a use as described herein, wherein the treatment comprises administering to the subject an amount of (+)-α-dihydrotetrabenazine of approximately 10 mg per day.
1.102 (+)-α-dihydrotetrabenazine for use, a method or a use as described herein, wherein the treatment comprises administering to the subject an amount of (+)-α-dihydrotetrabenazine of approximately 12.5 mg per day.
1.103 (+)-α-dihydrotetrabenazine for use, a method or a use as described herein, wherein the treatment comprises administering to the subject an amount of (+)-α-dihydrotetrabenazine of approximately 15 mg per day.

In each case, unless the context indicates otherwise, the quantity of (+)-α-dihydrotetrabenazine specified may be administered once per day or in several (e.g. two) doses per day.

In some embodiments, the quantity of (+)-α-dihydrotetrabenazine specified is administered once daily.

The administration of (+)-α-dihydrotetrabenazine typically forms part of a chronic treatment regime. The (+)-α-dihydrotetrabenazine may therefore be administered to a patient for a treatment period of at least a week, more usually at least two weeks, or at least a month, and typically longer than a month. Where a patient is shown to respond well to treatment, the period of treatment can be longer than six months and may extend over a period of years.

The chronic treatment regime may involve the administration of the (+)-α-dihydrotetrabenazine every day, or the treatment regime may include days when no (+)-α-dihydrotetrabenazine is administered.

The dosage administered to the subject may vary during the treatment period. For example, the initial dosage may be increased or decreased depending on the subject's response to the treatment. A subject may, for example, be given an initial low dose to test the subject's tolerance towards the (+)-α-dihydrotetrabenazine, and the dosage thereafter increased as necessary up to the maximum daily intake of 20 mg. Alternatively, an initial daily dosage administered to the patient may be selected so as to give an estimated desired degree of VMAT2 blockage, following which a lower maintenance dose may be given for the remainder of the treatment period, with the option of increasing the dosage should the subject's response to the treatment indicate that an increase is necessary.

The quantity of (+)-α-dihydrotetrabenazine required to achieve the desired therapeutic effect may be dependent on the weight of the subject to be treated. The quantities of (+)-α-dihydrotetrabenazine administered to the subject can be expressed in a number of mg/kg, wherein the kg relates the weight of the subject to be treated. The appropriate dosage amount can therefore be calculated by multiplying the mg/kg amount by the weight of the subject to be treated. Accordingly, the invention also provides:

1.104 (+)-α-dihydrotetrabenazine for use in a method for the treatment of a movement disorder, wherein the treatment comprises administering to a subject an amount of (+)-α-dihydrotetrabenazine between 0.01 mg/kg and 0.5 mg/kg per day provided that the total amount of (+)-α-dihydrotetrabenazine administered per day is in the range from 0.5 mg to 20 mg (e.g. 1 mg to 20 mg).
1.105 A method of treatment of a movement disorder in a subject in need thereof (e.g. a mammalian subject such as a human), which treatment comprises administering to the subject an amount of (+)-α-dihydrotetrabenazine between 0.01 mg/kg and 0.5 mg/kg per day, provided that the total amount of (+)-α-dihydrotetrabenazine administered per day is in the range from 0.5 mg to 20 mg (e.g. 1 mg to 20 mg).
1.106 The use of (+)-α-dihydrotetrabenazine for the manufacture of a medicament for the treatment of a movement disorder, which treatment comprises administering to the subject an amount of (+)-α-dihydrotetrabenazine between 0.01 mg/kg and 0.5 mg/kg, provided that the total amount of (+)-α-dihydrotetrabenazine administered per day is in the range from 0.5 mg to 20 mg (e.g. 1 mg to 20 mg).

In further embodiments, there is provided:

1.107 (+)-α-dihydrotetrabenazine for use, a method or a use as described herein, wherein the treatment comprises administering to the subject between 0.01 mg/kg and 0.3 mg/kg of (+)-α-dihydrotetrabenazine per day, provided that the total amount of (+)-α-dihydrotetrabenazine administered per day is in the range from 0.5 mg to 20 mg (e.g. 1 mg to 20 mg).
1.108 (+)-α-dihydrotetrabenazine for use, a method or a use as described herein, wherein the treatment comprises administering to the subject between 0.02 mg/kg and 0.3 mg/kg of (+)-α-dihydrotetrabenazine per day, provided that the total amount of (+)-α-dihydrotetrabenazine administered per day is in the range from 0.5 mg to 20 mg (e.g. 1 mg to 20 mg).
1.109 (+)-α-dihydrotetrabenazine for use, a method or a use as described herein, wherein the treatment comprises administering to the subject between 0.03 mg/kg and 0.3 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.110 (+)-α-dihydrotetrabenazine for use, a method or a use as described herein, wherein the treatment comprises administering to the subject between 0.04 mg/kg and 0.3 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.111 (+)-α-dihydrotetrabenazine for use, a method or a use as described herein, wherein the treatment comprises administering to the subject between 0.05 mg/kg and 0.3 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.112 (+)-α-dihydrotetrabenazine for use, a method or a use as described herein, wherein the treatment comprises administering to the subject between 0.02 mg/kg and 0.2 mg/kg of (+)-α-dihydrotetrabenazine per day, provided that the total amount of (+)-α-dihydrotetrabenazine administered per day is in the range from 0.5 mg to 20 mg (e.g. 1 mg to 20 mg).
1.113 (+)-α-dihydrotetrabenazine for use, a method or a use as described herein, wherein the treatment comprises administering to the subject between 0.03 mg/kg and 0.2 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.114 (+)-α-dihydrotetrabenazine for use, a method or a use as described herein, wherein the treatment comprises administering to the subject between 0.04 mg/kg and 0.2 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.115 (+)-α-dihydrotetrabenazine for use, a method or a use as described herein, wherein the treatment comprises administering to the subject between 0.05 mg/kg and 0.2 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.116 (+)-α-dihydrotetrabenazine for use, a method or a use as described herein, wherein the treatment comprises administering to the subject between 0.02 mg/kg and 0.1 mg/kg of (+)-α-dihydrotetrabenazine per day, provided that the total amount of (+)-α-dihydrotetrabenazine administered per day is in the range from 0.5 mg to 20 mg (e.g. 1 mg to 20 mg).
1.117 (+)-α-dihydrotetrabenazine for use, a method or a use as described herein, wherein the treatment comprises administering to the subject between 0.03 mg/kg and 0.1 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.118 (+)-α-dihydrotetrabenazine for use, a method or a use as described herein, wherein the treatment comprises administering to the subject between 0.04 mg/kg and 0.1 mg/kg of (+)-α-dihydrotetrabenazine, provided that the total amount of (+)-α-dihydrotetrabenazine administered per day is in the range from 0.5 mg to 20 mg (e.g. 1 mg to 20 mg).
1.119 (+)-α-dihydrotetrabenazine for use, a method or a use as described herein, wherein the treatment comprises administering to the subject between 0.05 mg/kg and 0.1 mg/kg of (+)-α-dihydrotetrabenazine, provided that the total amount of (+)-α-dihydrotetrabenazine administered per day is in the range from 0.5 mg to 20 mg (e.g. 1 mg to 20 mg).

In each of the foregoing aspects and embodiments, the (+)-α-dihydrotetrabenazine can be administered as the free base or as a pharmaceutically acceptable salt. In one embodiment, the (+)-α-dihydrotetrabenazine is administered as a pharmaceutically acceptable salt. In another embodiment, the (+)-α-dihydrotetrabenazine is administered as a free base. The quantities of (+)-α-dihydrotetrabenazine are calculated as the amounts of the free base, or when the (+)-α-dihydrotetrabenazine is in the form of a pharmaceutically acceptable salt, the amount of (+)-α-dihydrotetrabenazine free base present in the pharmaceutically acceptable salt.

The present inventors have found that plasma levels of (+)-α-dihydrotetrabenazine required for effective treatment of hyperkinetic movement disorders can be considerably lower than the plasma levels achieved by administration of Valbenazine as described in WO 2015/171802.

Accordingly, in a further aspect (1.120), the invention provides:
(+)-α-dihydrotetrabenazine, or a pharmaceutically acceptable salt thereof, for use in a method of treatment of a movement disorder; or
A method of treatment of a movement disorder in a subject in need thereof (e.g. a mammalian subject such as a human); or
The use of (+)-α-dihydrotetrabenazine for the manufacture of a medicament for the treatment of a movement disorder
   wherein the treatment comprises administering to a subject a therapeutically effective amount of the (+)-α-dihydrotetrabenazine or pharmaceutically acceptable salt thereof, in an amount sufficient to achieve an average blood plasma C_{avg} concentration, where measured over the period of five hours in the range from 2 ng/ml to 15 ng/ml.

In one embodiment (1.121), the invention provides:
(+)-α-dihydrotetrabenazine, or a pharmaceutically acceptable salt thereof, for use in a method of treatment of a movement; or
A method of treatment of a movement disorder in a subject in need thereof (e.g. a mammalian subject such as a human); or
The use of (+)-α-dihydrotetrabenazine for the manufacture of a medicament for the treatment of a movement disorder
   wherein the treatment comprises administering to a subject a therapeutically effective amount of the (+)-α-dihydrotetrabenazine or pharmaceutically acceptable salt thereof, in an amount sufficient to achieve an average blood plasma C_{avg} concentration, when measured over a period of three hours, in the range from 3 ng/ml to 15 ng/ml.

Complete blocking of VMAT2 is considered undesirable as this can lead to unwanted side effects, such as Parkinsonism. The present invention provides plasma levels of (+)-α-dihydrotetrabenazine that are sufficient to give effective treatment of movement disorders but do not block VMAT2 to an extent that causes Parkinsonism and similar side effects. The levels of VMAT2 blocking can be determined by competitive binding studies using Positron Emission Tomography (PET). By co-administering a radioactive ligand with the compound of interest at various concentrations, the proportion of binding sites occupied can be determined (see for example, Matthews et al., “Positron emission tomography molecular imaging for drug development”, Br. J. Clin. Pharmacol., 73:2, 175-186). Accordingly, the invention also provides:

1.122 (+)-α-dihydrotetrabenazine for use in a method for the treatment of a movement disorder, wherein the treatment comprises administering to a subject an amount of (+)-α-dihydrotetrabenazine sufficient to cause a level of blocking of up to 90% of the VMAT2 proteins in the subject.
1.123 A method of treatment of a movement disorder in a subject in need thereof (e.g. a mammalian subject such as a human), which treatment comprises administering to the subject an amount of (+)-α-dihydrotetrabenazine sufficient to cause a level of blocking of up to 90% of the VMAT2 proteins in the subject.
1.124 The use of (+)-α-dihydrotetrabenazine for the manufacture of a medicament for the treatment of a movement disorder, which treatment comprises administering to the subject an amount of (+)-α-dihydrotetrabenazine sufficient to cause a level of blocking of up to 90% of the VMAT2 proteins in the subject.

In further embodiments, there is provided:

1.125 (+)-α-dihydrotetrabenazine for use, a method or a use as described herein, wherein the treatment comprises administering to the subject an amount of (+)-α-dihydrotetrabenazine sufficient to cause a level of blocking of up to 85% of the VMAT2 proteins in the subject.
1.126 (+)-α-dihydrotetrabenazine for use, a method or a use as described herein, wherein the treatment comprises administering to the subject an amount of (+)-α-dihydrotetrabenazine sufficient to cause a level of blocking of up to 80% of the VMAT2 proteins in the subject.
1.127 (+)-α-dihydrotetrabenazine for use, a method or a use as described herein, wherein the treatment comprises administering to the subject an amount of (+)-α-dihydrotetrabenazine sufficient to cause a level of blocking of up to 75% of the VMAT2 proteins in the subject.
1.128 (+)-α-dihydrotetrabenazine for use, a method or a use as described herein, wherein the treatment comprises administering to the subject an amount of (+)-α-dihydrotetrabenazine sufficient to cause a level of blocking of up to 70% of the VMAT2 proteins in the subject.
1.129 (+)-α-dihydrotetrabenazine for use, a method or a use as described herein, wherein the treatment comprises administering to the subject an amount of (+)-α-dihydrotetrabenazine sufficient to cause a level of blocking of from 25% to 85% of the VMAT2 proteins in the subject.
1.130 (+)-α-dihydrotetrabenazine for use, a method or a use as described herein, wherein the treatment comprises administering to the subject an amount of (+)-α-dihydrotetrabenazine sufficient to cause a level of blocking of from 30% to 80% of the VMAT2 proteins in the subject.
1.131 (+)-α-dihydrotetrabenazine for use, a method or a use as described herein, wherein the treatment comprises administering to the subject an amount of (+)-α-dihydrotetrabenazine sufficient to cause a level of blocking of from 35% to 75% of the VMAT2 proteins in the subject.
1.132 (+)-α-dihydrotetrabenazine for use, a method or a use as described herein, wherein the treatment comprises administering to the subject an amount of (+)-α-dihydrotetrabenazine sufficient to cause a level of blocking of from 35% to 70% of the VMAT2 proteins in the subject.
1.133 (+)-α-dihydrotetrabenazine for use, a method or a use as described herein, wherein the treatment comprises administering to the subject an amount of (+)-α-dihydrotetrabenazine sufficient to cause a level of blocking of from 40% to 75% of the VMAT2 proteins in the subject.
1.134 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level of blocking of from 45% to 75% of the VMAT2 proteins in the subject.
1.135 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level of blocking of from 35% to 80% of the VMAT2 proteins in the subject.
1.136 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level of blocking of from 40% to 80% of the VMAT2 proteins in the subject.
1.137 (+)-α-dihydrotetrabenazine for use, a method or a use as described herein, wherein the treatment comprises administering to the subject an amount of (+)-α-dihydrotetrabenazine sufficient to cause a level of blocking of from 45% to 80% of the VMAT2 proteins in the subject.
1.138 (+)-α-dihydrotetrabenazine for use, a method or a use as described herein, wherein the treatment comprises administering to the subject an amount of (+)-α-dihydrotetrabenazine sufficient to cause a level of blocking of from 50% to 80% of the VMAT2 proteins in the subject.
1.139 (+)-α-dihydrotetrabenazine for use, a method or a use as described herein, wherein the treatment comprises administering to the subject an amount of (+)-α-dihydrotetrabenazine sufficient to cause a level of blocking of from 55% to 80% of the VMAT2 proteins in the subject.
1.140 (+)-α-dihydrotetrabenazine for use, a method or a use as described herein, wherein the treatment comprises administering to the subject an amount of (+)-α-dihydrotetrabenazine sufficient to cause a level of blocking of VMAT2 proteins in the subject of between 30% and 70%.
1.141 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a blocking level of VMAT2 proteins in the subject of between 30% and 65%.
1.142 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a blocking level of VMAT2 proteins in the subject of between 30% and 60%.
1.143 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level blocking of VMAT2 proteins in the subject of between 40% and 80%.
1.144 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level of blocking of VMAT2 proteins in the subject of between 40% and 75%.
1.145 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level of blocking of VMAT2 proteins in the subject of between 40% and 70%.
1.146 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level of blocking of VMAT2 proteins in the subject of between 40% and 65%.
1.147 (+)-α-dihydrotetrabenazine for use, a method or a use as described herein, wherein the treatment comprises administering to the subject in need thereof, wherein the method comprising administering to a subject an amount of (+)-α-dihydrotetrabenazine sufficient to cause a level blocking of VMAT2 proteins in the subject of between 40% and 60%.

In further embodiments of the invention, particular, it is envisaged that low doses of (+)-α-dihydrotetrabenazine may be used in the treatment of hyperkinetic movement disorders in juvenile human subjects aged from 5 years to 16 years old.

Accordingly, the invention also provides:

1.148 (+)-α-dihydrotetrabenazine for use in a method for the treatment of a movement disorder in a human subject aged from 5 years to 16 years old, wherein the treatment comprises administering to a subject an amount of (+)-α-dihydrotetrabenzine from 0.5 mg to 12.5 mg per day.
1.149 A method of treatment of a movement disorder in a human subject aged from 5 years to 16 years old in need thereof (e.g. a mammalian subject such as a human), which treatment comprises administering to the subject an amount of (+)-α-dihydrotetrabenazine from 0.5 mg to 12.5 mg per day.
1.150 The use of (+)-α-dihydrotetrabenazine for the manufacture of a medicament for the treatment of a movement disorder, which treatment comprises administering to a human subject aged from 5 years to 16 years old an amount of (+)-α-dihydrotetrabenazine from 0.5 mg to 12.5 mg per day.

In further embodiments, there is provided

1.151 (+)-α-dihydrotetrabenazine for use, a method or a use as defined in any one of Embodiments 1.148 to 1.150, wherein the treatment comprises administering to a subject an amount of (+)-α-dihydrotetrabenzine from 0.5 mg to 10 mg per day
1.152 (+)-α-dihydrotetrabenazine for use, a method or a use as defined in any one of Embodiments 1.148 to 1.150, wherein the treatment comprises administering to the subject an amount of (+)-α-dihydrotetrabenazine from 0.5 mg to 8 mg per day.
1.153 (+)-α-dihydrotetrabenazine for use, a method or a use as defined in any one of Embodiments 1.148 to 1.150, wherein the treatment comprises administering to the subject an amount of (+)-α-dihydrotetrabenazine from 0.5 mg to 7.5 mg per day.
1.154 (+)-α-dihydrotetrabenazine for use, a method or a use as defined in any one of Embodiments 1.148 to 1.150, wherein the treatment comprises administering to the subject an amount of (+)-α-dihydrotetrabenazine from 0.5 mg to 7 mg per day.
1.155 (+)-α-dihydrotetrabenazine for use, a method or a use as defined in any one of Embodiments 1.148 to 1.150, wherein the treatment comprises administering to the subject an amount of (+)-α-dihydrotetrabenazine from 0.5 mg to 6 mg per day.
1.156 (+)-α-dihydrotetrabenazine for use, a method or a use, as defined in any one of Embodiments 1.148 to 1.150, wherein the treatment comprises administering to the subject an amount of (+)-α-dihydrotetrabenazine from 0.5 mg to 5 mg per day.
1.157 (+)-α-dihydrotetrabenazine for use, a method or a use as defined in any one of Embodiments 1.148 to 1.150, wherein the treatment comprises administering to the subject an amount of (+)-α-dihydrotetrabenazine from 1 mg to 12.5 mg per day.
1.158 (+)-α-dihydrotetrabenazine for use, a method or a use as defined in any one of Embodiments 1.148 to 1.150, wherein the treatment comprises administering to a subject an amount of (+)-α-dihydrotetrabenzine from 1 mg to 10 mg per day
1.159 (+)-α-dihydrotetrabenazine for use, a method or a use as defined in any one of Embodiments 1.148 to 1.150, wherein the treatment comprises administering to the subject an amount of (+)-α-dihydrotetrabenazine from 1 mg to 8 mg per day.
1.160 (+)-α-dihydrotetrabenazine for use, a method or a use as defined in any one of Embodiments 1.148 to 1.150, wherein the treatment comprises administering to the subject an amount of (+)-α-dihydrotetrabenazine from 1 mg to 7.5 mg per day.
1.161 (+)-α-dihydrotetrabenazine for use, a method or a use as defined in any one of Embodiments 1.148 to 1.150, wherein the treatment comprises administering to the subject an amount of (+)-α-dihydrotetrabenazine from 1 mg to 7 mg per day.
1.162 (+)-α-dihydrotetrabenazine for use, a method or a use as defined in any one of Embodiments 1.148 to 1.150, wherein the treatment comprises administering to the subject an amount of (+)-α-dihydrotetrabenazine from 1 mg to 6 mg per day.
1.163 (+)-α-dihydrotetrabenazine for use, a method or a use as defined in any one of Embodiments 1.148 to 1.150, wherein the treatment comprises administering to the subject an amount of (+)-α-dihydrotetrabenazine from 1 mg to 5 mg per day.
1.164 (+)-α-dihydrotetrabenazine for use, a method or a use as defined in any one of Embodiments 1.148 to 1.150, wherein the treatment comprises administering to the subject an amount of (+)-α-dihydrotetrabenazine of approximately 2.5 mg per day.
1.165 (+)-α-dihydrotetrabenazine for use, a method or a use as defined in any one of Embodiments 1.148 to 1.150, wherein the treatment comprises administering to the subject an amount of (+)-α-dihydrotetrabenazine of approximately 5 mg per day.
1.166 (+)-α-dihydrotetrabenazine for use, a method or a use as defined in any one of Embodiments 1.148 to 1.150, wherein the treatment comprises administering to the subject an amount of (+)-α-dihydrotetrabenazine of approximately 7.5 mg per day.
1.167 (+)-α-dihydrotetrabenazine for use, a method or a use as defined in any one of Embodiments 1.148 to 1.150, wherein the treatment comprises administering to the subject an amount of (+)-α-dihydrotetrabenazine of approximately 10 mg per day.

In each of Embodiments 1.148 to 1.167, the human subjects to be treated with (+)-α-dihydrotetrabenazine are from 5 years old to 16 years old. The subjects typically have a weight of 80 kg or less, for example a weight in the range from 17 kg to 70 kg.

In some embodiments (Embodiment 1.168), the (+)-α-dihydrotetrabenazine is administered to juveniles having a weight from 20 to 65 kg.

In other embodiments (Embodiment 1.169), the (+)-α-dihydrotetrabenazine is administered to juveniles having a weight from 20 to 60 kg

In one embodiment (Embodiment 1.170), the subjects are aged from 5 years to 8 years old and the (+)-α-dihydrotetrabenazine is administered in an amount of 0.5 mg to 5 mg per day.

In another embodiment (Embodiment 1.171), the subjects are aged from 9 years to 12 years old and the (+)-α-dihydrotetrabenazine is administered in an amount of 1 mg to 8 mg per day

In a further embodiment (Embodiment 1.172), the subjects are aged from 13 years to 16 years old and the (+)-α-dihydrotetrabenazine is administered in an amount of 2 mg to 12.5 mg per day.

In a further embodiment (Embodiment 1.173) the (+)-α-dihydrotetrabenazine for use, a method or a use as defined in any one of Embodiments 1.148 to 1.172, may comprise steps (a), (b) and optionally (c), (d), (e) and (f) as defined in any one of Embodiments 1.1 to 1.36.

In the methods and uses of each of Embodiments 1.1 to 1.52B and 1.85 to 1.173, the amounts of (+)-α-dihydrotetrabenazine or a pharmaceutically acceptable salt required to be administered to a subject may depend in part on the speed of metabolism of the compound by the subject.

Metabolism of many drugs (approximately 25% of all current prescription drugs) involves the cytochrome P450 2D6 (CYP2D6) enzyme which is primarily expressed in the liver.

Approximately 7% of the population has a slow acting form of this enzyme and 7% a super-fast acting form. Thirty-five percent are carriers of a non-functional CYP2D6 allele.

As a result, subjects can be classified according to whether they are:
poor metabolizers—with little or no CYP2D6 function;
intermediate metabolizers—who metabolize drugs at a rate somewhere between the poor and extensive metabolizers;
extensive metabolizers—with normal CYP2D6 function; or
ultrarapid metabolizer—with multiple copies of the CYP2D6 gene are expressed, so greater-than-normal CYP2D6 function occurs.

Thus, considerable variation exists in the efficiency and amount of CYP2D6 enzyme produced between individuals. Hence, for drugs that are metabolized by CYP2D6, certain individuals will eliminate these drugs quickly (ultrarapid metabolizers) while others will do so slowly (poor metabolizers). If a drug is metabolized too quickly, it may decrease the drug's efficacy while if the drug is metabolized too slowly, toxicity may result.

A number of commercially available diagnostic tests are available for determining whether a subject is a fast or slow metabolizer and these include the CYP2D6 Genotyping tests available from Genelex Labs LLC, Seattle, Wash. 98121, USA, and Trimgen Corporation, Sparks, Maryland 21152, USA.

Therefore, in each of the methods and uses described herein, for example in Embodiments 1.1 to 1.52B and 1.85 to 1.173, the use or method may include the step of testing a subject to determine the CYP2D6 status of the subject and then using the outcome of the test as a factor in determining the amounts of (+)-α-dihydrotetrabenazine or a pharmaceutically acceptable salt to be administered to the subject. Thus, a fast metabolizer may be administered an amount towards the upper end of a given range whereas a slower metabolizer may be administered a smaller amount.

In each of the foregoing embodiments 1.1 to 1.173, the unit dosage form (or the substance administered in the method) contains no more than 20% by weight, relative to the (+)-α-dihydrotetrabenazine, of any other isomer of dihydrotetrabenazine; more usually contains no more than 10% by weight, relative to the (+)-α-dihydrotetrabenazine, of any other isomer of dihydrotetrabenazine; preferably contains no more than 5% by weight, relative to the (+)-α-dihydrotetrabenazine, of any other isomer of dihydrotetrabenazine; and more preferably contains no more than 2% by weight, relative to the (+)-α-dihydrotetrabenazine, of any other isomer of dihydrotetrabenazine.

Thus, the (+)-α-dihydrotetrabenazine typically has an isomeric purity of at least 80%.

The term “isomeric purity” in the present context refers to the amount (+)-α-dihydrotetrabenazine present relative to the total amount or concentration of dihydrotetrabenazines of all isomeric forms. For example, if 90% of the total dihydrotetrabenazine present in the composition is (+)-α-dihydrotetrabenazine then the isomeric purity is 90%.

The (+)-α-dihydrotetrabenazine of the invention may have an isomeric purity of greater than 82%, greater than 85%, greater than 87%, greater than 90%, greater than 91%, greater than 92%, greater than 93%, greater than 94%, greater than 95%, greater than 96%, greater than 97%, greater than 98%, greater than 99%, greater than 99.5%, or greater than 99.9%.

### Salts

The dihydrotetrabenazine may be presented as the free base or in the form of salts. All references herein to (+)-α-dihydrotetrabenazine include (+)-α-dihydrotetrabenazine in both free base and salt forms thereof, unless the context indicates otherwise.

In one embodiment (Embodiment 1.174), the (+)-α-dihydrotetrabenazine as defined in any one of Embodiments 1.1 to 1.173 is in the form of the free base.

In another embodiment (Embodiment 1.175), the (+)-α-dihydrotetrabenazine as defined in any one of Embodiments 1.1 to 1.173 is in the form of a salt.

The salts are typically acid addition salts.

The salts can be synthesized from the parent compound by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free base form of the compound with the acid in water or in an organic solvent, or in a mixture of the two; generally, nonaqueous media such as ether, ethyl acetate, ethanol, isopropanol, or acetonitrile are used.

Acid addition salts may be formed with a wide variety of acids, both inorganic and organic. Examples of acid addition salts include salts formed with an acid selected from the group consisting of acetic, 2,2-dichloroacetic, adipic, alginic, ascorbic (e.g. L-ascorbic), L-aspartic, benzenesulphonic, benzoic, 4-acetamidobenzoic, butanoic, (+) camphoric, camphor-sulphonic, (+)-(1S)-camphor-10-sulphonic, capric, caproic, caprylic, cinnamic, citric, cyclamic, dodecylsulphuric, ethane-1,2-disulphonic, ethanesulphonic, 2-hydroxyethanesulphonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrobromic, hydrochloric, hydriodic, isethionic, (+)-L-lactic, (±)-DL-lactic, lactobionic, maleic, malic, (−)-L-malic, malonic, (±)-DL-mandelic, methanesulphonic, naphthalene-2-sulphonic, naphthalene-1,5-disulphonic, 1-hydroxy-2-naphthoic, nicotinic, nitric, oleic, orotic, oxalic, palmitic, pamoic, phosphoric, propionic, L-pyroglutamic, salicylic, 4-amino-salicylic, sebacic, stearic, succinic, sulphuric, tannic, (+)-L-tartaric, thiocyanic, p-toluenesulphonic, undecylenic and valeric acids, as well as acylated amino acids and cation exchange resins.

The salt forms of the compound of the invention are typically pharmaceutically acceptable salts, and examples of pharmaceutically acceptable salts are discussed in Berge et al., 1977, “Pharmaceutically Acceptable Salts,” J. Pharm. Sci., Vol. 66, pp. 1-19. However, salts that are not pharmaceutically acceptable may also be prepared as intermediate forms which may then be converted into pharmaceutically acceptable salts. Such non-pharmaceutically acceptable salts forms, which may be useful, for example, in the purification or separation of the compounds of the invention, also form part of the invention.

### Isotopes

The (+)-α-dihydrotetrabenazine may contain one or more isotopic substitutions, and a reference to a particular element includes within its scope all isotopes of the element. For example, a reference to hydrogen includes within its scope ¹H, ²H (D), and ³H (T). Similarly, references to carbon and oxygen include within their scope respectively ¹¹C, ¹²C, ¹³C and ¹⁴C and ¹⁶O and ¹⁸O.

Typically, the (+)-α-dihydrotetrabenazine of the invention does not contain isotopes (such as ¹¹C or ³H) in amounts higher than their natural abundance.

In one embodiment, the percentage of the total hydrogen atoms in the (+)-α-dihydrotetrabenazine that are deuterium atoms is less than 2%, more typically less than 1%, more usually less than 0.1%, preferably less than 0.05% and most preferably no more than 0.02%.

In an analogous manner, a reference to a particular functional group also includes within its scope isotopic variations, unless the context indicates otherwise.

The isotopes may be radioactive or non-radioactive. In one embodiment of the invention, the (+)-α-dihydrotetrabenazine contains no radioactive isotopes. Such compounds are preferred for therapeutic use. In another embodiment, however, the (+)-α-dihydrotetrabenazine may contain one or more radioisotopes. Compounds containing such radioisotopes may be useful in a diagnostic context.

### Solvates

(+)-α-Dihydrotetrabenazine may form solvates.

Examples of solvates are solvates formed by the incorporation into the solid state structure (e.g. crystal structure) of the compounds of the invention of molecules of a non-toxic pharmaceutically acceptable solvent (referred to below as the solvating solvent). Examples of such solvents include water, alcohols (such as ethanol, isopropanol and butanol) and dimethylsulphoxide. Solvates can be prepared by recrystallising the compounds of the invention with a solvent or mixture of solvents containing the solvating solvent. Whether or not a solvate has been formed in any given instance can be determined by subjecting crystals of the compound to analysis using well known and standard techniques such as thermogravimetric analysis (TGE), differential scanning calorimetry (DSC) and X-ray crystallography.

The solvates can be stoichiometric or non-stoichiometric solvates.

Particular solvates are hydrates, and particular examples of hydrates include hemihydrates, monohydrates and dihydrates.

For a more detailed discussion of solvates and the methods used to make and characterise them, see Bryn et al., Solid-State Chemistry of Drugs, Second Edition, published by SSCI, Inc of West Lafayette, Ind., USA, 1999, ISBN 0-967-06710-3.

Alternatively, rather than existing as a hydrate, the compound of the invention may be anhydrous. Therefore, in another embodiment, the (+)-α-dihydrotetrabenazine is in an anhydrous form.

### Methods for the Preparation of (+)-α-Dihydrotetrabenazine

(+)-α-Dihydrotetrabenazine (compound of formula (I)) can be prepared from tetrabenazine according to the synthetic route shown in Scheme 1.

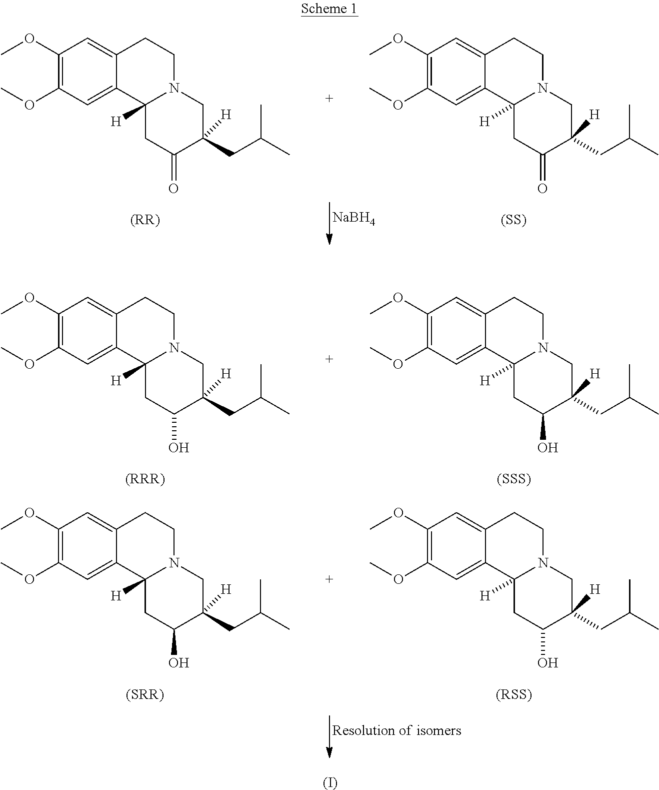

Racemic tetrabenazine (3-isobutyl-9,10-dimethoxy-1,3,4,6,7,11b-hexahydro-2H-pyrido[2,1,a]isoquinolin-2-one) containing the RR and SS isomers of tetrabenazine is reduced with sodium borohydride to afford a mixture of four dihydrotetrabenazine isomers of which a racemic mixture of the α-dihydrotetrabenazines (RRR and SSS isomers) constitutes the major product and a racemic mixture of the β-dihydrotetrabenazines (the SRR and RSS isomers) constitutes a minor product. The β-dihydrotetrabenazines can be removed during an initial purification procedure, for example by chromatography or recrystallization and then the racemic α-dihydrotetrabenazines resolved (e.g. by recrystallisation with di-p-toluoyl-L-tartaric acid or (R)-(−)-camphorsulfonic acid or by chiral chromatography), to afford (+)-α-dihydrotetrabenazine (I) ((2R,3R,11bR)-3-isobutul-9,10-dimethox-1,3,4,6,7,11b-hexahydro-2H-pyrido[2,1,a]isoquinolin-2-01). The stereochemical configuration of (+)-α-dihydrotetrabenazine can be determined, so example by forming a salt such as the mesylate salt in crystalline form and the structure identified by X-ray crystallography.

(+)-α-Dihydrotetrabenazine can also be prepared according to Yao et al., “Preparation and evaluation of tetrabenazine enantiomers and all eight stereoisomers of dihydrotetrabenazine as VMAT2 inhibitors”, Eur. J. Med. Chem., (2011), 46, pp. 1841-1848.

### Pharmaceutical Compositions

In each of Embodiments 1.1 to 1.175, where the compound is administered as a pharmaceutical unit dosage form, or other pharmaceutical composition, the pharmaceutical compositions can be in any form suitable for oral, parenteral, topical, intranasal, intrabronchial, ophthalmic, otic, rectal, intra-vaginal, or transdermal administration. Where the compositions are intended for parenteral administration, they can be formulated for intravenous, intramuscular, intraperitoneal, subcutaneous administration or for direct delivery into a target organ or tissue by injection, infusion or other means of delivery.

Pharmaceutical dosage forms suitable for oral administration include tablets, capsules, caplets, pills, lozenges, syrups, solutions, sprays, powders, granules, elixirs and suspensions, sublingual tablets, sprays, wafers or patches and buccal patches. In one embodiment, the dosage form is a tablet. In another embodiment, the dosage form is a capsule.

A particular group of pharmaceutical dosage forms for use in accordance with Embodiments 1.1 to 1.175 consists of tablets, capsules, solutions, syrups, suspensions and gels.

Pharmaceutical compositions containing the dihydrotetrabenazine compound of the invention can be formulated in accordance with known techniques, see for example, Remington's Pharmaceutical Sciences, Mack Publishing Company, Easton, Pa., USA.

Thus, tablet compositions can contain a unit dosage of active compound together with an inert diluent or carrier such as a sugar or sugar alcohol, e.g.; lactose, sucrose, sorbitol or mannitol; and/or a non-sugar derived diluent such as sodium carbonate, calcium phosphate, talc, calcium carbonate, or a cellulose or derivative thereof such as methyl cellulose, ethyl cellulose, hydroxypropyl methyl cellulose, and starches such as corn starch. Tablets may also contain such standard ingredients as binding and granulating agents such as polyvinylpyrrolidone, disintegrants (e.g. swellable crosslinked polymers such as crosslinked carboxymethylcellulose), lubricating agents (e.g. stearates), preservatives (e.g. parabens), antioxidants (e.g. BHT), buffering agents (for example phosphate or citrate buffers), and effervescent agents such as citrate/bicarbonate mixtures. Such excipients are well known and do not need to be discussed in detail here.

Capsule formulations may be of the hard gelatin or soft gelatin variety and can contain the active component in solid, semi-solid, or liquid form. Gelatin capsules can be formed from animal gelatin or synthetic or plant derived equivalents thereof.

The solid dosage forms (e.g.; tablets, capsules etc.) can be coated or un-coated, but typically have a coating, for example a protective film coating (e.g. a wax or varnish) or a release controlling coating. The coating (e.g. a Eudragit™ type polymer) can be designed to release (+)-α-dihydrotetrabenazine at a desired location within the gastro-intestinal tract. Thus, the coating can be selected so as to degrade under certain pH conditions within the gastrointestinal tract, thereby selectively release the compound in the stomach or in the ileum or duodenum.

The release controlling coating can be designed to release (+)-α-dihydrotetrabenazine at such as rate that a therapeutically effective plasma concentration is maintained for at least 2 hours, more typically at least 3 hours, for example at least 4 hours, or at least 5 hours.

The composition may comprise a multiplicity of individual units such as pellets or minitablets which each contain (+)-α-dihydrotetrabenazine and which may each be coated with a release controlling agent.

Instead of, or in addition to, a coating, the drug can be presented in a solid matrix comprising a release controlling agent, for example a release delaying agent which may be adapted to selectively release the compound under conditions of varying acidity or alkalinity in the gastrointestinal tract. Alternatively, the matrix material or release retarding coating can take the form of an erodible polymer (e.g. a maleic anhydride polymer) which is substantially continuously eroded as the dosage form passes through the gastrointestinal tract.

Compositions for topical use include ointments, creams, sprays, patches, gels, liquid drops and inserts (for example intraocular inserts). Such compositions can be formulated in accordance with known methods.

Compositions for parenteral administration are typically presented as sterile aqueous or oily solutions or fine suspensions, or may be provided in finely divided sterile powder form for making up extemporaneously with sterile water for injection.

Examples of formulations for rectal or intra-vaginal administration include pessaries and suppositories which may be, for example, formed from a shaped mouldable or waxy material containing the active compound.

Compositions for administration by inhalation may take the form of inhalable powder compositions or liquid or powder sprays, and can be administrated in standard form using powder inhaler devices or aerosol dispensing devices. Such devices are well known. For administration by inhalation, the powdered formulations typically comprise the active compound together with an inert solid powdered diluent such as lactose.

Particular pharmaceutical compositions of the invention are compositions selected from:
Sublingual compositions;
Intranasal;
Pellets or tablets formulated to provide release kinetics corresponding to zero order release of the active compound;
Pellets or tablets formulated to provide first fast release followed by constant rate release (zero order) of the active compound;
Pellets or tablets formulated to provide a mixture of first order and zero order release of the active compound; and
Pellets or tablets formulated to provide a combination of zero order and first order release of the active compound; and optionally a further order of release of the active compound selected from second, third and fourth orders of release and combinations thereof.

Pellets and tablets formulated to provide release kinetics of the types defined above can be prepared according to methods well known the skilled person; for example as described in Remington's Pharmaceutical Sciences (idem) and “Remington—The Science and Practice of Pharmacy, 21^{st} edition, 2006, ISBN 0-7817-4673-6.

The compounds of the inventions will generally be presented in unit dosage form and, as such, will typically contain sufficient compound to provide a desired level of biological activity. Such amounts are set out above.

The active compound will be administered to a subject (patient) in need thereof (for example a human or animal patient) in an amount sufficient to achieve the desired therapeutic effect.

### BRIEF DESCRIPTION OF THE DRAWINGS

FIG. 1 is a plot of % VMAT2 binding vs body weight after administration of doses of 7.5 mg, 15 mg and 22.5 mg of (+)-α-dihydrotetrabenazine to human subjects.

FIG. 2 is a plot of % VMAT2 binding vs amounts of (+)-α-dihydrotetrabenazine administered to human subjects in mg/kg body weight.

FIG. 3 shows the average total distance traveled by rats when treated with vehicle (with or without amphetamine induction) and (+)-α-dihydrotetrabenazine at doses of 0.5, 1, 1.5 and 2 mg/kg and risperidone at a dose of 1 mg/kg in amphetamine-induced rats, as described in Example 2, Study 1 below.

FIG. 4 shows the average total stereotypic behaviour by rats when treated with vehicle (with or without amphetamine induction) and (+)-α-dihydrotetrabenazine at doses of 0.5, 1, 1.5 and 2 mg/kg and risperidone at a dose of 1 mg/kg in amphetamine-induced rats, as described in Example 2, Study 1 below.

FIG. 5 shows the average total distance traveled by rats when treated with vehicle (with or without amphetamine induction) and (+)-α-dihydrotetrabenazine at doses of 0.1 mg/kg and 0.25 mg/kg and risperidone at a dose of 1 mg/kg in amphetamine-induced rats, as described in Example 2, Study 2 below.

FIG. 6 shows the average total stereotypic behaviour by rats when treated with vehicle (with or without amphetamine induction) and (+)-α-dihydrotetrabenazine at doses of 0.1 mg/kg and 0.25 mg/kg and risperidone at a dose of 1 mg/kg in amphetamine-induced rats, as described in Example 2, Study 2 below.

FIG. 7 shows the average total distance traveled by rats when treated with vehicle and (+)-α-dihydrotetrabenazine at a dose of 2.5 mg/kg or 5 mg/kg and risperidone at a dose of 1 mg/kg in rats without amphetamine induction, as described in Example 2, Study 3 below.

FIG. 8 shows the average total stereotypic behaviour by rats when treated with vehicle and (+)-α-dihydrotetrabenazine at a dose of 2.5 mg/kg or 5 mg/kg and risperidone at a dose of 1 mg/kg in rats without amphetamine induction, as described in Example 2, Study 3 below.

FIG. 9 shows the average total distance traveled by rats when treated with vehicle and (+)-α-dihydrotetrabenazine and Valbenazine each at a dose of 1 mg/kg or 1.5 mg/kg and risperidone at a dose of 1 mg/kg in amphetamine-induced rats, as described in Example 2, Study 4 below.

FIG. 10 shows the average total stereotypic behaviour by rats when treated with vehicle and (+)-α-dihydrotetrabenazine and Valbenazine each at a dose of 1 mg/kg or 1.5 mg/kg and risperidone at a dose of 1 mg/kg in amphetamine-induced rats, as described in Example 2, Study 4 below.

### EXAMPLES

The following non-limiting examples illustrate the synthesis and properties of the (+)-alpha-dihydrotetrabenazine salt of the invention.

### Example 1

(+)-α-Dihydrotetrabenazine in defined amounts was administered by oral dosing to five human volunteers. In four of the volunteers, blood sample were taken at 30, 60, 120 and 180 minutes after drug administration. Blood samples were not taken from the fifth volunteer. At 60 minutes after drug administration, PET scans were initiated and these were stopped at 120 minutes after drug administration.

The experiment was carried out at dosages of 7.5 mg, 15 mg and 22.5 mg.

### Results

Table 1 shows the plasma concentrations in nanogrammes/ml of (+)-α-dihydrotetrabenazine in five human subjects, 0.5, 1, 1.5, 2 and 3 hours after a dose of 7.5 mg, 15 mg and 22.5 mg. Table 2 shows the % VMAT2 blocking following administration of 7.5 mg, 15 mg and 22.5 mg of (+)-α-dihydrotetrabenazine in all five subjects.

TABLE 1

| | Time | Subject # | | | | | |
|---|---|---|---|---|---|---|---|
| | (h) | 1 | 2 | 3 | 4 | 5 | 6 |
| Body | | 112 | 76 | 129 | 59 | 91 | 68 |
| Weight | | | | | | | |
| (kg) | | | | | | | |
| Dose | | | | | | | |
| (oral) | | | | | | | |
| 7.5 mg | 0.5 | BLQ | 0.531 | 0.216 | 8.43 | 6.68 | BLQ |
| | 1 | 0.94 | 13.7 | 4.35 | 15.0 | 9.8 | 3.77 |
| | 1.5 | 2.39 | 10.8 | 6.91 | 20.7 | 13.5 | 3.06 |
| | 2 | 2.44 | 14.0 | 5.03 | 17.6 | 10.7 | 4.33 |
| | 3 | 3.01 | 22.2 | 6.96 | 19.6 | 11.2 | 9.18 |
| 15 mg | 0.5 | 4.02 | 7.99 | 1.2 | 26.7 | 15.6 | 5.41 |
| | 1 | 11.1 | 22.8 | 14.3 | 53.8 | 34.2 | 10.6 |
| | 1.5 | 10.7 | 46.4 | 17.9 | 42.5 | 27.1 | 16.1 |
| | 2 | 10.2 | 35.7 | 12.0 | 53.3 | 31.8 | 17.4 |
| | 3 | 10.6 | 46.5 | 18.2 | 60.2 | 27.1 | 14.9 |
| 22.5 mg | 0.5 | 9.61 | 5.23 | 9.04 | ND | 55.3 | 5.58 |
| | 1 | 18.0 | 21.8 | 34.7 | ND | 45.1 | 8.01 |
| | 1.5 | 16.8 | 36.2 | 29.8 | ND | 46.2 | 5.87 |
| | 2 | 14.9 | 40.2 | 26.3 | ND | 41.9 | 12.5 |
| | 3 | 13.2 | 51.8 | 17.3 | ND | 42.5 | 18.7 |

BLQ—Below level of quantitation,
ND—Not done

TABLE 2

| | | Subject # | | | | | |
|---|---|---|---|---|---|---|---|
| | | 1 | 2 | 3 | 4 | 5 | 6 |
| Body | | 112 | 76 | 129 | 59 | 91 | 68 |
| Weight | | | | | | | |
| (kg) | | | | | | | |
| Dose | 7.5 mg | 54 | 73 | 62 | 84 | 73 | 60 |
| (oral) | | | | | | | |
| | 15 mg | 73 | 83 | 69 | 89 | 79 | 76 |
| | 22.5 mg | 75 | 82 | 74 | ND | 82 | 62 |

Although in subjects with a lower body weight, higher (+)-α-dihydrotetrabenazine blood plasma concentrations were observed for a given dose, it can be seen that even in heavier individuals, at least 50% % VMAT2 blocking was observed at doses as low as 7.5 mg and, in lighter individuals, significantly higher % VMAT2 binding was. It was also observed that during the period of PET scanning, average plasma levels of less than 15 ng/ml gave rise to % VMAT2 binding of at least 50%.

The data demonstrate that very low doses of (+)-α-dihydrotetrabenazine resulting in plasma concentrations of less than 15 ng/ml can still give high levels of VMAT2 blocking.

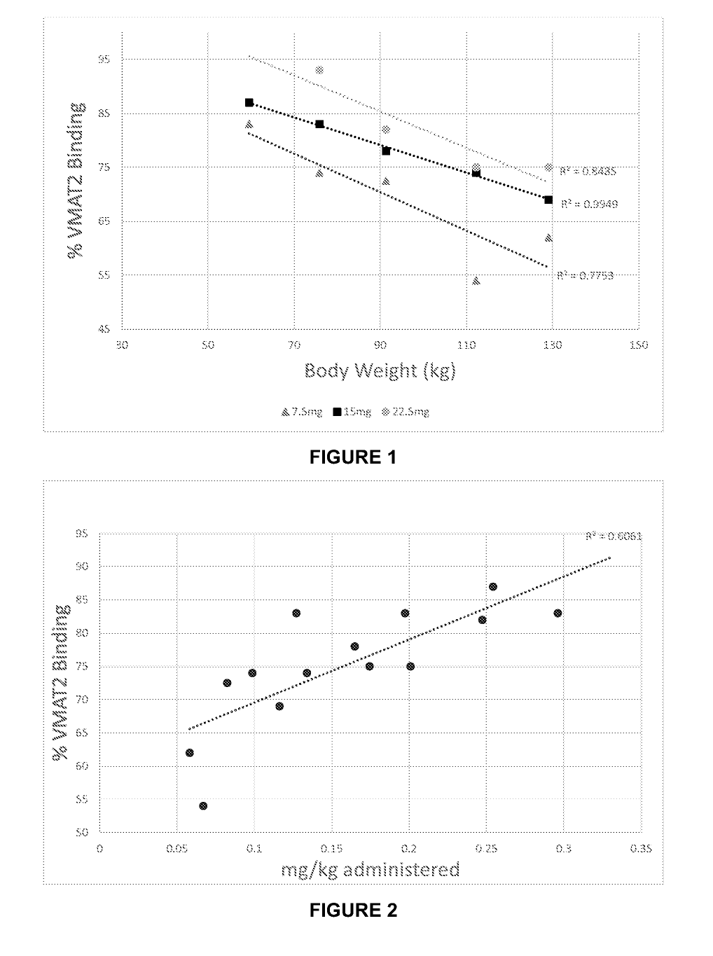

The data for subjects 1 to 5 were used to prepare plots (FIG. 1) of % VMAT2 binding against body weight at the three dosage levels and plots (FIG. 2) of % VMAt2 binding against the amount (in mg/kg body weight) of (+)-α-dihydrotetrabenazine administered. The data for subject 6, which are somewhat anomalous, were not included in FIG. 1 or FIG. 2.

FIG. 1 shows the % VMAT2 binding against body weight for each dose of (+)-α-DHTBZ administered (7.5 mg, 15 mg and 22.5 mg) based on the data above. As can be seen, for a given dose there is a good correlation between body weight and % VMAT2 binding.

FIG. 2 shows the % VMAT2 binding against the amount of (+)-α-DHTBZ per kg of the body weight of the subject based on the data above. Again it can be seen that there is a good correlation between amount of (+)-α-DHTBZ per weight of the subject (mg/kg value) and % VMAT2 binding.

Therefore, it is expected that the amount of (+)-α-DHTBZ that needs to be administered to provide a given VMAT2 binding level will depend greatly on the weight of the subject.

### Example 2—Comparison of the Effect of Dihydrotetrabenazines and Risperidone on Amphetamine-Induced Hyperlocomotion

Dopaminergic models for Tourette's syndrome use systemic or focal administration of dopamine agonists such as amphetamine. After injection with amphetamine, a test animal expresses stereotypic behaviour. In particular, involvement of a dopaminergic system implicated in Tourette's syndrome wild type mice and rats can be stimulated with amphetamine and the resulting hyperactivity and stereotypes can be reversed with dopamine antagonists such as risperidone and haloperidol (Tourette's syndrome—Animal Models for Screening, Charles River Discovery Research Services, Finland).

Amphetamine produced a rise in extracellular concentrations of brain dopamine and concomitant behavioural manifestations in the rat and other species. At relatively low doses (1.2 ng/kg i.p.) amphetamine increases locomotor behaviour, ceases movement and gives way to a stationary posture accompanied by highly repetitive rapid head movements. This latter non-locomotor phase of stimulation is referred to as focused stereotypy. The stereotypy can last for over an hour and is usually followed by a period of locomotor stimulation (Schiorring 1971).

Administration of dopamine agonists (such as amphetamine) is known to induce behavioural stereotypes and sensorimotor gating disruption. Also, dopaminergic, cholinergic (TANs) and HDC models (subsequent to stress and/or amphetamine injection) are known to show an increase in stereotypic behaviours (Yaol et al 2016).

Amphetamine induced stereotype behaviour has also been evaluated as a model for the movement disorder condition, tardive dyskinesia (see Rubovitis et al (1972)).

The atypical antipsychotic drug risperidone is commonly used for the treatment of Tourette's syndrome and has been described (J. D. Walkup, A Guide to Tourette Syndrome Medications, Publ. 2008, The National Tourette Syndrome Association, Inc.) as being probably the best atypical antipsychotic for tic suppression with potentially less risk of motor side effects than haloperidol and fluphenazine.

Three studies were carried out to compare the effects of dihydrotetrabenazines and risperidone on amphetamine-induced and non-amphetamine-induced hyperlocomotion in rats, on the basis that, for the reasons given above, locomotor studies are useful models for Tourette's syndrome and other movement disorders.

### Materials and Methods

### Equipment

Open field arena, Med Associates Inc.
Plastic syringes 1 ml, Terumo. Ref: SS-01T1
Animal feeding needle 15 G, Instech Solomon, Cat: 72-4446

### Sartorius Mechatronics Scale A22101, Sartorius Weighting Technology, Germany

### Needle 27 G Terumo Myjector, 0.5 ml, Ref: 8300010463

Plastic syringes 3 ml, Soft-Ject, Ref: 8300005761
BD Microtainer K2EDTA tubes Ref: 365975

### Matrix 0.75 ml, Alphanum Tubes, Thermo Scientific, Ref: 4274

Microplate Devices, Uniplate 24 wells, 10 ml, Ref: 734-1217
Thermo Electron Corp. Heraeus Fresco 17, refrigerated centrifuge

### Test Animals

All animal experiments were carried out according to the National Institute of Health (NIH) guidelines for the care and use of laboratory animals, and approved by the National Animal Experiment Board, Finland. Male CD (Charles River Laboratories, Germany) at weight range of 200-250 g (165-200 g upon arrival) were used for the experiments. Animals were housed at a standard temperature (22±1° C.) and in a light-controlled environment (lights on from 7 am to 8 pm) with ad libitum access to food and water.

### Methods

Locomotor activity of the rats was tested in open field arena. The open field test was performed during the rat light cycle and under a normal lighting evenly distributed to the test chambers. The paths of the rats were recorded by activity monitor (Med. Associates Inc.).

Dosing the vehicle, vehicle-amphetamine, (+)-α-DHTBZ or risperidone was done prior to LMA test. The rats were placed in the centre of the arena, and the path was recorded for 60 minutes.

### Endpoint, Blood Samples and Tissue Processing

Within 10 minutes from the end of the test animals were euthanized by an overdose of CO₂. The terminal blood sample was collected with cardiac puncture from all compound treated rats from each group excluding vehicle rats. 0.5 ml of blood was collected with syringe attached to 18 G needle and moved into precooled K2-EDTA microtubes. The EDTA microtube was inverted several times to mix up the EDTA and blood. Tubes were then immediately put on wet ice and centrifuged (Heraeus Fresco 17) within 10-15 minutes of collecting (9.6×1000 G/10×1000 RPM, +4° C. for 2 min), and 200 μl of plasma was collected in 96-tube plates (Matrix Technologies ScreenMates 0.75 ml Alphanumeric Round-Bottom Storage tubes, PP) on dry ice according to sample map.

After collection of blood the neck was dislocated at the base of the skull. Brain was collected and weighed. Brain weights were recorded and the brain was frozen on dry ice on the 24 well plate.

The plasma and brain samples were stored at −80° C. until sent for analysis or destroyed.

### Study 1

The effects on stereotypic behaviour and the distance traveled in rats following administration of (+)-α-dihydrotetrabenazine dosed at 0.5 mg/kg to 2 mg/kg, as well as risperidone at 1 mg/kg, were studied.

Animals were grouped as follows:
Group 1: 10 rats treated with Vehicle (t=0 min) and Vehicle (t=30 min)
Group 2: 10 rats treated with Vehicle (t=0 min) and Amphetamine (t=30 min)
Group 3: 10 rats treated with (+)-α-DHTBZ 0.5 mg/kg (t=0 min) and Amphetamine (t=30 min)
Group 4: 10 rats treated with (+)-α-DHTBZ 1 mg/kg (t=0 min) and Amphetamine (t=30 min)
Group 5: 10 rats treated with (+)-α-DHTBZ 1.5 mg/kg (t=0 min) and Amphetamine (t=30 min)
Group 6: 10 rats treated with (+)-α-DHTBZ 2 mg/kg (t=0 min) and Amphetamine (t=30 min)
Group 7: 10 rats treated with risperidone 1 mg/kg (t=0 min) and Amphetamine (t=30 min)

### Results

### 1. Distance Traveled

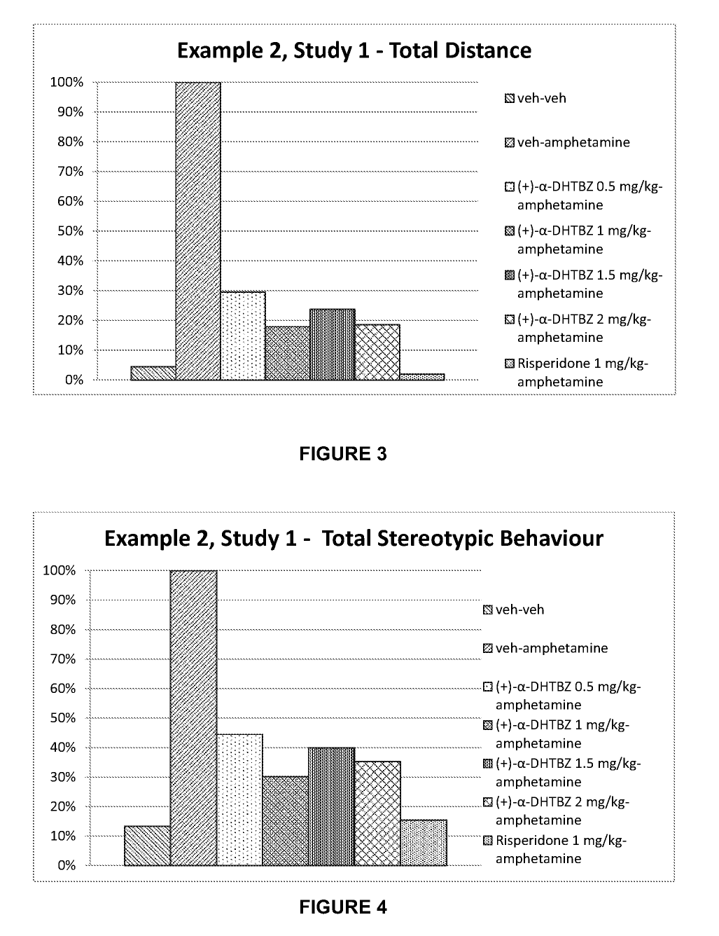

Rats dosed with either vehicle, (+)-α-DHTBZ 0.5 mg/kg, (+)-α-DHTBZ 1 mg/kg, (+)-α-DHTBZ 1.5 mg/kg, (+)-α-DHTBZ 2 mg/kg or Risperidone 1 mg/kg were subjected to LMA testing first for 30 min and then for 60 minutes after vehicle or amphetamine challenge. Resulting locomotor activity was evaluated in 3 min bins and as a total over the testing period. The normalised total distance traveled over the testing time is presented in FIG. 3.

When compared to the vehicle-vehicle group the vehicle-amphetamine was significantly different. When compared to vehicle-amphetamine group the vehicle-vehicle, (+)-α-DHTBZ 0.5 mg/kg, (+)-α-DHTBZ 1 mg/kg, (+)-α-DHTBZ 1.5 mg/kg, (+)-α-DHTBZ 2 mg/kg and risperidone 1 mg/kg were significantly different.

### 2. Stereotypic Behaviour

Rats dosed with either vehicle, (+)-α-DHTBZ 0.5 mg/kg, (+)-α-DHTBZ 1 mg/kg, (+)-α-DHTBZ 1.5 mg/kg, (+)-α-DHTBZ 2 mg/kg or Risperidone 1 mg/kg were subjected to LMA testing first for 30 min and then for 60 minutes after vehicle or amphetamine challenge. Resulting stereotypic activity was evaluated in 3 min bins and as a total over the testing period. The normalised total stereotypic behaviour over the testing time is presented in FIG. 4.

When compared to the vehicle-vehicle group the vehicle-amphetamine, (+)-α-DHTBZ 0.5 mg/kg and (+)-α-DHTBZ 1.5 mg/kg were significantly different. When compared to vehicle-amphetamine group the vehicle-vehicle, (+)-α-DHTBZ 0.5 mg/kg, (+)-α-DHTBZ 1 mg/kg, (+)-α-DHTBZ 1.5 mg/kg, (+)-α-DHTBZ 2 mg/kg and risperidone 1 mg/kg were significantly different.

### Conclusions

This study evaluated the effect of (+)-α-DHTBZ at doses 0.5 mg/kg, 1 mg/kg, 1.5 mg/kg and 2 mg/kg and risperidone at dose 1 mg/kg on amphetamine induced locomotor activity in male CD rats.

(+)-α-DHTBZ at all the tested doses and risperidone 1 mg/kg led to lower locomotor activity when compared to the vehicle-amphetamine group. (+)-α-DHTBZ at all the tested doses and risperidone 1 mg/kg led to reduced stereotypic behaviour when compared to the vehicle-amphetamine group. Both of the measured parameters suggest that (+)-α-DHTBZ has a sedative effect similar to risperidone.

### Study 2

The effects on stereotypic behaviour and the distance traveled in rats following administration of (+)-α-dihydrotetrabenazine dosed at 0.1 mg/kg to 0.25 mg/kg, as well as risperidone at 1 mg/kg, were studied.

Animals were grouped as follows:
Group 1: 10 rats treated with Vehicle (t=0 min) and Vehicle (t=30 min)
Group 2: 10 rats treated with Vehicle (t=0 min) and Amphetamine (t=30 min)
Group 3: 10 rats treated with (+)-α-DHTBZ 0.1 mg/kg (t=0 min) and Amphetamine (t=30 min)
Group 4: 10 rats treated with (+)-α-DHTBZ 0.25 mg/kg (t=0 min) and Amphetamine (t=30 min)
Group 5: 10 rats treated with risperidone 1 mg/kg (t=0 min) and Amphetamine (t=30 min)

### Results

### 1 Distance Traveled

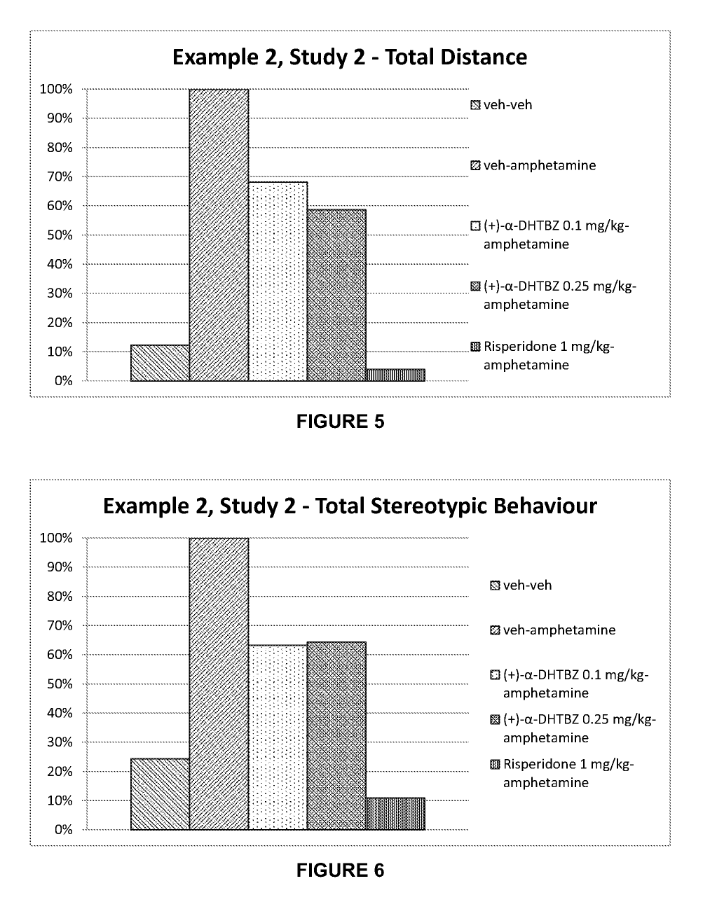

Rats dosed with either vehicle, (+)-α-DHTBZ 0.1 mg/kg, (+)-α-DHTBZ 0.25 mg/kg, or Risperidone 1 mg/kg were subjected to LMA testing first for 30 min and then for 60 minutes after vehicle or amphetamine challenge. Resulting locomotor activity was evaluated in 3 min bins and as a total over the testing period. The normalised total distance traveled over the testing time is presented in FIG. 5.

When compared to vehicle-amphetamine group the vehicle-vehicle, (+)-α-DHTBZ 0.25 mg/kg and risperidone 1 mg/kg were significantly different.

### 2 Stereotypic Behaviour

Rats dosed with either vehicle, (+)-α-DHTBZ 0.1 mg/kg, (+)-α-DHTBZ 0.25 mg/kg, or Risperidone 1 mg/kg were subjected to LMA testing first for 30 min and then for 60 minutes after vehicle or amphetamine challenge. Resulting stereotypic activity was evaluated in 3 min bins and as a total over the testing period. The normalised total stereotypic behaviour over the testing time is presented in FIG. 6.

When compared to vehicle-amphetamine group the vehicle-vehicle, (+)-α-DHTBZ 0.1 mg/kg, (+)-α-DHTBZ 0.25 mg/kg and risperidone 1 mg/kg were significantly different.

### Conclusions

This study evaluated the effect of (+)-α-DHTBZ at doses of 0.1 mg/kg and 0.25 mg/kg and risperidone at dose 1 mg/kg on amphetamine induced locomotor activity in male CD rats.

(+)-α-DHTBZ at 0.25 mg/kg and risperidone 1 mg/kg led to lower locomotor activity when compared to the vehicle-amphetamine group. (+)-α-DHTBZ at both the tested doses and risperidone 1 mg/kg led to reduced stereotypic behaviour when compared to the vehicle-amphetamine group.

### Study 3

The effects of (+)-α-dihydrotetrabenazine and risperidone on in non-amphetamine induced rats was studied. Animals were grouped as follows:
Group 1: 10 rats treated with Vehicle
Group 2: 10 rats treated with (+)-α-DHTBZ 2.5 mg/kg
Group 3: 10 rats treated with (+)-α-DHTBZ 5 mg/kg
Group 4: 10 rats treated with risperidone 1 mg/kg

### Results

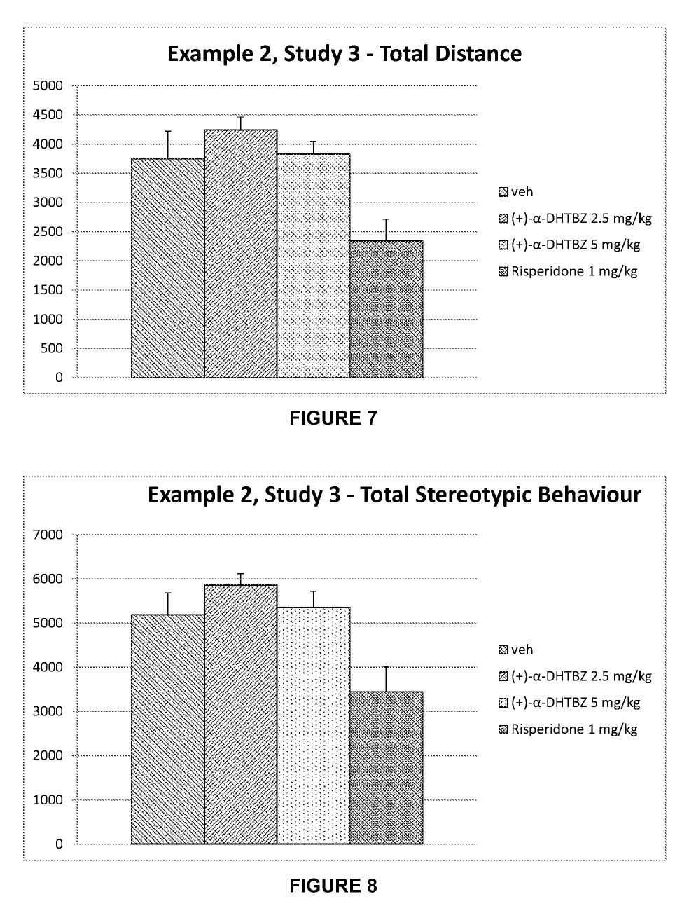

In non-induced rats, the total movement and stereotypic behaviour in rats treated with the vehicle were comparable to (+)-α-dihydrotetrabenazine (see FIGS. 7 and 8). However, rats treated with risperidone had reduced total movement and reduced total stereotypic behaviour.

### Study 4

The effects on stereotypic behaviour and the distance traveled in rats following administration of (+)-α-dihydrotetrabenazine and Valbenazine both dosed at 1 mg/kg and 1.5 mg/kg, as well as risperidone at 1 mg/kg, were studied.

Animals were grouped as follows:
Group 1: 10 rats treated with Vehicle (t=0 min) and Vehicle (t=30 min)
Group 2: 10 rats treated with Vehicle (t=0 min) and Amphetamine (t=30 min)
Group 3: 10 rats treated with (+)-α-DHTBZ 1 mg/kg (t=0 min) and Amphetamine (t=30 min)
Group 4: 10 rats treated with (+)-α-DHTBZ 1.5 mg/kg (t=0 min) and Amphetamine (t=30 min)
Group 5: 10 rats treated with Valbenazine 1 mg/kg (t=0 min) and Amphetamine (t=30 min)
Group 6: 10 rats treated with Valbenazine 1.5 mg/kg (t=0 min) and Amphetamine (t=30 min)
Group 7: 10 rats treated with risperidone 1 mg/kg (t=0 min) and Amphetamine (t=30 min)

### Results

### 1 Distance Traveled

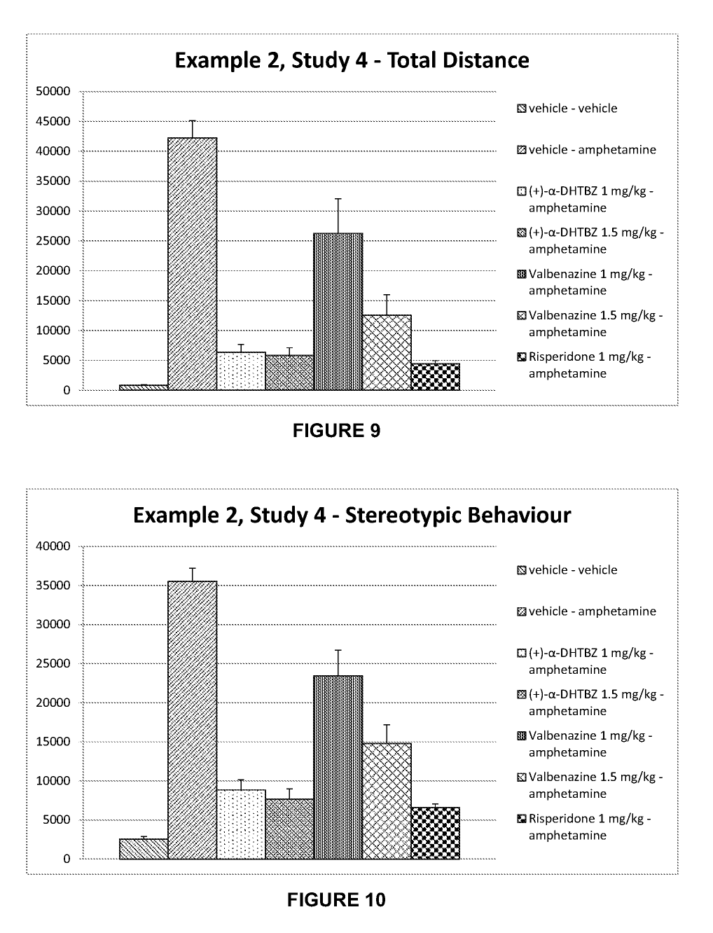

Rats dosed with either vehicle, (+)-α-DHTBZ, Valbenazine or Risperidone were subjected to LMA testing first for 30 min and then for 60 minutes after vehicle or amphetamine challenge. Resulting locomotor activity was evaluated in 3 min bins and as a total over the testing period. The total distance traveled over the testing time is presented in FIG. 9.

When compared to vehicle-amphetamine group the vehicle-vehicle, (+)-α-DHTBZ (at 1 mg/kg and 1.5 mg/kg), Valbenazine (at 1 mg/kg and 1.5 mg/kg) and risperidone 1 mg/kg were significantly different.

### 2 Stereotypic Behaviour

Rats dosed with either vehicle, (+)-α-DHTBZ 0.1 mg/kg, (+)-α-DHTBZ 0.25 mg/kg, or Risperidone 1 mg/kg were subjected to LMA testing first for 30 min and then for 60 minutes after vehicle or amphetamine challenge. Resulting stereotypic activity was evaluated in 3 min bins and as a total over the testing period. The normalised total stereotypic behaviour over the testing time is presented in FIG. 10.

When compared to vehicle-amphetamine group the vehicle-vehicle, (+)-α-DHTBZ (at 1 mg/kg and 1.5 mg/kg), Valbenazine (at 1 mg/kg and 1.5 mg/kg) and risperidone 1 mg/kg were significantly different.

### Conclusions

This study evaluated the effect of (+)-α-DHTBZ and Valbenazine both at doses of 1 mg/kg and 1.5 mg/kg and risperidone at dose 1 mg/kg on amphetamine induced locomotor activity in male CD rats.

(+)-α-DHTBZ at 1 mg/kg and 1.5 mg/kg led to lower locomotor activity when compared to the vehicle-amphetamine group and the corresponding dose of Valbenazine. (+)-α-DHTBZ at both the tested doses led to reduced stereotypic behaviour when compared to the vehicle-amphetamine group and the corresponding dose of Valbenazine.

### Comments

Studies 1 and 2 in Example 2 show the effectiveness of doses of (+)-α-dihydrotetrabenazine as low as 0.1 mg/kg in reducing movement in amphetamine-induced rats. It is therefore expected that such low dose regimes may also be useful in treating hyperkinetic movement disorders in humans.

Study 3 in Example 2 suggests that following administration of low doses of (+)-α-dihydrotetrabenazine whereas abnormal movements of the type found in movement disorders will be reduced or suppressed by the drug, normal movements will not be. This is in contrast to risperidone, a well-used treatment for movement disorders, where the levels of both normal and abnormal movements can be reduced by administration of the drug.

Study 4 in Example 2 shows the increased effectiveness of (+)-α-dihydrotetrabenazine over Valbenazine.

### EQUIVALENTS

It will readily be apparent that numerous modifications and alterations may be made to the specific embodiments of the invention described above without departing from the principles underlying the invention. All such modifications and alterations are intended to be embraced by this application.

1. A method of treating a movement disorder in a subject in need thereof, which treatment comprises the steps of:
(a) administering to the subject an initial daily dosage of (+)-α-dihydrotetrabenazine or pharmaceutically acceptable salt thereof, wherein the initial daily dosage is an amount of (+)-α-dihydrotetrabenazine or pharmaceutically acceptable salt thereof corresponding to from 0.5 mg to 5 mg of (+)-α-dihydrotetrabenazine free base;
(b) carrying out a clinical evaluation of the subject for efficacy and side effects arising from the treatment;
(c) where the clinical evaluation (b) has established that an increased daily dosage of (+)-α-dihydrotetrabenazine or pharmaceutically acceptable salt thereof is desirable, administering an increased daily dosage which is greater than the initial daily dosage by an incremental amount of (+)-α-dihydrotetrabenazine or pharmaceutically acceptable salt thereof corresponding to from 0.5 mg to 5 mg of (+)-α-dihydrotetrabenazine free base; or, where the clinical evaluation has established that an increased daily dosage is not desirable, either maintaining the initial daily dosage, reducing the dosage, or discontinuing the treatment;
(d) where an increased daily dosage has been administered, carrying out a further clinical evaluation of the subject for efficacy and side effects arising from the treatment with the increased daily dosage;
(e) where the further clinical evaluation (d) has established that a further increased daily dosage of (+)-α-dihydrotetrabenazine or pharmaceutically acceptable salt thereof is desirable, administering a further increased daily dosage which is greater than an immediately preceding daily dosage by an incremental amount of (+)-α-dihydrotetrabenazine or pharmaceutically acceptable salt thereof corresponding to from 0.5 mg to 5 mg of (+)-α-dihydrotetrabenazine free base; or, where the clinical evaluation has established that a further increased daily dosage is not desirable, maintaining the immediately preceding daily dosage, reducing the immediately preceding dosage or discontinuing the treatment; and
(f) optionally repeating steps (d) and (e) as often as desired until an optimum daily dosage is reached.

2. A method according to claim 1 wherein the initial daily dosage of (+)-α-dihydrotetrabenazine or pharmaceutically acceptable salt thereof, is an amount corresponding to from 0.5 mg to 3 mg of (+)-α-dihydrotetrabenazine free base.

3. A method according to claim 2 wherein the initial daily dosage of (+)-α-dihydrotetrabenazine or pharmaceutically acceptable salt thereof, is an amount corresponding to 0.5 mg, 1 mg, 1.5 mg, or 2 mg of (+)-α-dihydrotetrabenazine free base.

4. A method according to claim 1 wherein the increased daily dosage in step (c) is an amount which is greater than the initial daily dosage by an incremental amount of (+)-α-dihydrotetrabenazine or pharmaceutically acceptable salt thereof corresponding to from 0.5 mg to 3 mg of (+)-α-dihydrotetrabenazine free base.

5. A method according to claim 4 wherein the increased daily dosage in step (c) is an amount which is greater than the initial daily dosage by an incremental amount of (+)-α-dihydrotetrabenazine or pharmaceutically acceptable salt thereof corresponding to 0.5 mg, 1 mg, 1.5 mg, or 2 mg of (+)-α-dihydrotetrabenazine free base.

6. A method according to claim 1 wherein the further increased daily dosage in step (e) is greater than an immediately preceding daily dosage by an incremental amount of (+)-α-dihydrotetrabenazine or pharmaceutically acceptable salt thereof corresponding to from 0.5 mg to 3 mg of (+)-α-dihydrotetrabenazine free base.

7. A method according to claim 6 wherein the further increased daily dosage in step (e) is greater than an immediately preceding daily dosage by an incremental amount of (+)-α-dihydrotetrabenazine or pharmaceutically acceptable salt thereof corresponding to 0.5 mg, 1 mg, 1.5 mg, or 2 mg of (+)-α-dihydrotetrabenazine free base.

8. A method according to claim 1 wherein the treatment comprises the administration of a maximum (e.g. optimized) daily dosage of (+)-α-dihydrotetrabenazine or pharmaceutically acceptable salt thereof, which is an amount corresponding to no greater than 20 mg of (+)-α-dihydrotetrabenazine free base.

9. A method according to claim 8 wherein the treatment comprises the administration of a maximum (e.g. optimized) daily dosage of (+)-α-dihydrotetrabenazine or pharmaceutically acceptable salt thereof, which is an amount corresponding to no greater than 10 mg of (+)-α-dihydrotetrabenazine free base.

10. A method according to claim 1 wherein the treatment comprises determining an approximate weight of the subject and:
(i) when the subject has a weight of 30 kg to 50 kg, administering a maximum (e.g. optimized) daily amount of (+)-α-dihydrotetrabenazine or a pharmaceutically acceptable salt thereof corresponding to from 2 mg to 7.5 mg of (+)-α-dihydrotetrabenazine free base;
(ii) when the subject has a weight of 50 kg to 75 kg, administering a maximum (e.g. optimized) daily amount of (+)-α-dihydrotetrabenazine or a pharmaceutically acceptable salt thereof corresponding to from 5 mg to 10 mg of (+)-α-dihydrotetrabenazine free base;
(iii) when the subject has a weight of 75 kg to 95 kg, administering a maximum (e.g. optimized) daily amount of (+)-α-dihydrotetrabenazine or a pharmaceutically acceptable salt thereof corresponding to from 7.5 mg to 15 mg of (+)-α-dihydrotetrabenazine free base; or
(iv) when the subject has a weight of greater than 95 kg, administering a maximum (e.g. optimized) daily amount of (+)-α-dihydrotetrabenazine or a pharmaceutically acceptable salt thereof corresponding to from 15 mg to 20 mg of (+)-α-dihydrotetrabenazine free base the amount of (+)-α-dihydrotetrabenazine administered per day is from 15 mg to 20 mg.

11. A method of treating a movement disorder in a subject in need thereof, which treatment comprises administering to the subject an effective amount of (+)-α-dihydrotetrabenazine or a pharmaceutically acceptable salt thereof, wherein the said effective amount corresponds to an amount of (+)-α-dihydrotetrabenazine free base of from 0.05 mg/kg to 0.3 mg/kg per day.

12. A method according to claim 1 wherein the daily dosage, daily amount or effective amount of (+)-α-dihydrotetrabenazine or a pharmaceutically acceptable salt thereof administered to the subject is sufficient to cause a level of blocking of between 50% and 85% of VMAT2 proteins in the subject.

13. A method according to claim 11 wherein the daily dosage, daily amount or effective amount of (+)-α-dihydrotetrabenazine or a pharmaceutically acceptable salt thereof administered to the subject is sufficient to cause a level of blocking of between 50% and 85% of VMAT2 proteins in the subject.

14. A method according to claim 1 wherein the movement disorder is selected from tardive dyskinesia, Tourette's syndrome and Huntington's disease.

15. A method according to claim 14 wherein the movement disorder is Tourette's Syndrome.

16. A method according to claim 14 wherein the movement disorder is Huntington's Disease.

17. A method according to claim 1 wherein the (+)-α-dihydrotetrabenazine or pharmaceutically acceptable salt thereof is administered to the subject once per day.

18. A method according to claim 1 wherein the (+)-α-dihydrotetrabenazine or pharmaceutically acceptable salt thereof is not administered in combination with a therapeutically effective amount of amantadine.

19. A method according to claim 1 wherein the subject is a human subject from 5 years old to 16 years old, having a weight of 80 kg or less, for example a weight in the range from 17 kg to 70 kg.

20. A unit dosage form comprising from 0.5 mg to 3 mg of (+)-α-dihydrotetrabenazine, or a pharmaceutically acceptable salt thereof, and a pharmaceutically acceptable excipient.
